(19) United States
(12) Patent Application Publication
(10) Pub. No.: US 2020/0009293 A1
(43) Pub. Date: Jan. 9, 2020

(54) ELASTIC BIORESORBABLE ENCASEMENT FOR IMPLANTS

(71) Applicant: ORCHID MEDICAL PTE LTD, Singapore (SG)

(72) Inventors: Koon Kiat Teu, Singapore (SG); Jingnan Luo, Singapore (SG); Honglei Wang, Singapore (SG)

(21) Appl. No.: 16/337,836

(22) PCT Filed: Sep. 27, 2017
(86) PCT No.: PCT/SG2017/050481

§ 371 Date: Mar. 28, 2019

(51) Int. Cl. A61L 27/34; A61L 27/54; A61L 29/08; A61L 29/16; A61L 31/10; C08L 67/04; C08L 77/04

(52) U.S. Cl. CPC A61L 27/34; A61L 27/54; A61L 29/085; A61L 29/16; C08L 2203/02; C08L 67/04; C08L 77/04; C08L 2201/06; A61L 31/10

(57) ABSTRACT

Disclosed herein are elastic, bioresorbable encasements for medical implants, methods for making the same and uses thereof.

### CROSS REFERENCE TO RELATED APPLICATIONS

The present application is a 371 of PCT Patent Application No. PCT/SG2017/050481, filed Sep. 27, 2017, which claims priority to U.S. Provisional Application No. 62/400,714, filed Sep. 28, 2016, both of which are incorporated herein by reference in their entireties.

### FIELD OF INVENTION

The present invention relates to an elastic biologically compatible resorbable article configured to encasement medical implants.

### BACKGROUND

The listing or discussion of an apparently prior-published document in this specification should not necessarily be taken as an acknowledgement that the document is part of the state of the art or is common general knowledge.

Various desirable features of an implant, such as an antibacterial effect and the promotion of bone growth or cell recovery are often introduced to the implant by the use of coatings on the surface of the implant. For example, the colonization of bacteria on the surface of implants often leads to infections. To combat such infections, systemic antibiotics have been used to attempt to reduce the risk of infection. However, even when a subject is treated with antibiotics in a systemic manner, it is still possible for infections to develop on the surface of the implants. While site-specific delivery of antibiotics can be an effective alternative, metal implants are not easily modified to comprise a drug release mechanism for a desired period of time. With that in mind, a coating impregnated with a drug or antibiotic may be applied to the surface of the implant appears to be a better way to combat infection.

Thus, functional coatings, in which biologically active agents are coated on the surface of implants, is a common way to provide the implants with specific features such as antimicrobial, bone growth or cell recovery. However, while the use of coatings on the surface of an implant can be seen to be useful, there remain significant challenges associated with such coatings. These challenges include whether the agents to be coated are suitable for such a use, the ability of the coating to adhere to the surface of the implant and providing a controlled release of the active agents within the coating itself. In addition, these variables may also be affected by the active agents(s) used within the coating, which can be, for example, analgesics, antineoplastic agents, bisphosphonates and growth promoting substances.

Methods for coating implants where the coats serve as drug carriers have been developed. For example, see Von Eiff et al. Infections Associated with Medical Devices. Drugs 2005; 65 (2): 179-214. However, these implant coatings tend to fail as the coatings are often too mechanically unstable to survive the procedure that inserts and fixes the implant to the desired site within a subject. In addition, pre-coated implants do not allow customization of the drug or drugs delivered via the implant as required by the specific facts surrounding the patient to be treated (e.g. the need for specific combinations of drugs).

Another practical problem with implants that are coated is that each coated implant represents a new product that is the subject of a separate regulatory submission and which must pass through many regulatory hurdles before it can be used in a clinical setting. This is because, even though the implant itself remains the same, the act of coating it with a new substance (even if just changing the active ingredient) means that the regulatory authority must validate the coating method, coating efficacy, packaging and sterilization methods. As a result, if a company wants to provide an implant that has a broad portfolio of coating options (to deal with specific issues faced by the patients), they will need to submit each coated implant as a separate product for regulatory approval, which is a major undertaking. This represents a significant logistical and financial challenge.

One potential solution to at least some of the problems described above is to provide an implant encasement that contains the desired biologically active materials. This would allow for a single implant to be encased in differing encasements depending on the circumstances of the patient to be treated, allowing easier customization.

Currently, site-specific implant encasements are limited to inelastic envelopes that carry implants that do not have a strict need for the encasement to match the shape of the implant, or to fit within the space of the implant site. Such implants that do not have such strict requirements include pacemakers. However, many implants do have strict space and shape requirements for ergonomics and functionality and so the current encasements, which are loose fitting and may not easily be accommodated within the implant space are unsuitable for use on most implants.

A current commercially available implant encasement that can carry antibiotics is the Medtronic Tyrx implant. The use of the Tryx implant is largely limited to cardiovascular implantable electronic devices (CI ED) and implantable neurosimulators (INS). The encasement provided by the Tyrx encasement is essentially in the form of an envelope or pouch. Envelope-type encasements are not suitable for implants (e.g. hip implants) where the overall implant needs to retain the shape of the implant for ergonomics and functionality. Envelope encasements are also not suitable for implants that are difficult to implant or have limited space requirements, such as orthopedic screws. As the envelope-type encasement has to be bigger than the implant (in order for the implant to fit inside it) and does not provide any grip on the implant, the implant can move within the envelope freely. Given this, the implantation site might need to be slightly enlarged to accommodate the extra space and material required for the use of the envelope encasement. As such implant encasements are very flexible, but inelastic, there have been efforts to make the encasement stiffer to make it easier to handle when attempting to place the implant inside the encasement. In addition, currently-developed envelope-type implants simply coat antimicrobial agents onto the surface of the envelope, which is not ideal because the drug layer may be easily damaged during implantation, reducing the effectiveness of the envelope at the desired site of implantation.

U.S. Pat. No. 8,900,620 describes a biologically-compatible sleeve, where the sleeve requires a closed end to ensure that an implant can be retained within it. The sleeve is made from a non-woven sheet of a resorbable polymer that comprises a drug impregnated into the polymer. Currently, there are no commercial products using the sleeve described in this patent. Based upon the disclosed materials used in the patent, there is very little (if any) grip force from the encasement on the implant—otherwise there would be no need to have a second end that is closed to ensure that the medical implant is retained. While the patent mentions that the polymeric materials used can be stretched to enable the encasement to encapsulate an implant, there is no discussion of said materials being elastic (nor are the materials mentioned in embodiments considered to be elastic), so that they are able to recover at least part of their original dimensions. Therefore, the encasement appears to suffer from similar problems to those described above for envelope encasements, such as the Tyrx encasement.

Thus, there remains a need for improved implant encasements that enable the delivery of active agents to the desired site of action, whether said agents are anti-microbial in nature or are other active agents, such as analgesics, antineoplastic agents, bisphosphonates and growth promoting substances.

### SUMMARY OF INVENTION

It has been surprisingly found that an elastic medical implant encasement can be used to solve many of the problems disclosed hereinbefore. Said elastic medical implant encasement can:
carry different agents
be bioresorbed; and
be used on a wide range of implants.

Thus, the elastic medical implant encasement of the current invention is a significant milestone for many surgical procedures, which can now include an encasement, while previously it was not possible to do so.

Aspects and embodiments of the current disclosure are described with reference to the numbered clauses hereinbelow.

1. An elastic medical implant encasement, comprising:
at least one sheet of elastic material configured to form an encasement for at least part of a medical implant; and
at least one biologically active substance in at least one region of the at least one sheet of elastic material, wherein
the at least one sheet of elastic material comprises at least one polymer that is biologically-compatible and resorbable and has an elastic recovery of from 80% to 100% following stretching, or can stretch from its original size to an expanded size and return to its original size or to a size no greater than the expanded size minus 80% of the difference between expanded size and original size, optionally wherein the encasement or film can stretch from its original size to an expanded size and return to its original size or to a size no greater than the expanded size minus 90% of the difference between expanded size and original size.

2. The encasement of Clause 1, wherein the encasement is in the form of a tube, an envelope, a body comprising one or more anchoring portions, a film comprising two or more anchoring points, or a combination of any of these forms.

3. The encasement of Clause 1 or Clause 2, wherein the encasement comprises:
(a) at least one sheet of elastic material with two or more anchoring points formed via folding onto itself or with at least one additional sheet of elastic material, where at least one of the elastic material sheets carries at least one biologically active substance in at least one region;
(b) at least one sheet of elastic biologically-compatible, resorbable, material folded onto itself to form a single large anchoring surface, where the at least one elastic material sheet carries at least one biologically active substance in at least one region;
(c) at least two sheets of elastic material sealed at overlapping areas to form one or more anchoring points or surfaces, where at least one of the elastic material sheets carries at least one biologically active substance in at least one region; or
(d) the encasement comprises a seamless tubular structure formed from at least one sheet of elastic material, where the at least one elastic material sheet carries at least one biologically active substance in at least one region.

4. The encasement of any one of the preceding clauses, wherein the biologically active substance is encapsulated within the at least one sheet of elastic material and/or is coated on the surface of the at least one sheet of elastic material.

5. The encasement of any one of the preceding clauses, wherein the at least one sheet of elastic material is from two to ten sheets of elastic material.

6. The encasement of Clause 5, wherein the biologically active substance is encapsulated within one or more (e.g. one) of the two to ten sheets of elastic material and/or is coated on the surface of one or more (e.g. one) of the two to ten sheets of elastic material, optionally wherein the coated surface is not an outer surface of the two to ten sheets of elastic material.

7. The encasement of any one of the preceding clauses, wherein the one or more elastic sheets are configured to release the at least one biologically active substance at at least one releasing rate.

8. The encasement of any one of the preceding clauses, wherein the at least one sheet of elastic material has a total thickness of from 0.01 μm to 1000 μm.

9. The encasement of any one of the preceding clauses, wherein the at least one polymer is selected from one or more of the group consisting of poly(lactide-co-caprolactone), poly(DL-lactide-co-caprolactone) (DL-PLCL), poly(L-lactide-co-caprolactone) (PLLCL)polycaprolactone (PCL), polyglycolide (PGA), poly(L-lactic acid) (PLLA), poly(glycolide-co-caprolactone) (PGCL) copolymer, poly(D,L-lactic acid), poly(L-lactide-co-D, L-lactide) (PLDLLA), poly(L-lactide-co-glycolide) (PLGA), poly(D,L-lactide-co-glycolide), poly (D-lactide) (PDLA), poly(trimethylene carbonate) (PTMC), poly(lactide-co-trimethylene carbonate) (PLTMC), poly(gycolide-trimethylene carbonate), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone-co-butylacrylate, a copolymer of polyhydroxybutyrate, a copolymer of maleic anhydride, a copolymer of poly(trimethylene carbonate), polyethylene glycol, hydroxypropylmethylcellulose and cellulose derivatives, polysaccharides, such as hyaluronic acid, chitosan, starch, proteins such as gelatin, collagen or PEG derivatives.

10. The encasement of any one of the preceding clauses, wherein the number average molecular weight of the polymer is greater than 10,000 Daltons.

11. The encasement of any one of the preceding clauses, wherein
a) the at least one polymer is poly(lactide-co-caprolactone) (PLCL) (e.g. having a PLA to PCL ratio of from 90:10 to 60:40) or its derivatives and copolymers thereof;

and/or
b) the at least one polymer is poly(DL-lactide-co-caprolactone) (DL-PLCL) (e.g. having a DL-PLA to PCL ratio of from 90:10 to 50:50) or its derivatives and copolymers thereof; and/or
c) the at least one polymer is poly(glycolide-co-caprolactone) (PGCL) (e.g. having a PGA to PCL ratio of from 90:10 to 10:90) or its derivatives and copolymers thereof; and/or
d) the at least one polymer is a blend of PLCL or DL-PLCL or PGCL with a releasing agent selected from one or more of the group selected from polysorbate 20, polysorbate 40, polysorbate 60, polysorbate 80, or polyethyleneglycol having a molecular weight of 200 to 2000 Daltons in a w wt ratio of PLCL or DL-PLCL or PGCL to releasing agent of from 25:1 to 1:9.

12. The encasement of any one of the preceding clauses, wherein the biologically active substance is selected from one or more of the group consisting of an adrenocorticostatic, a β-adrenolytic, an androgen or antiandrogen, an antianemic, an antiparasitic, an anabolic, an anaesthetic or analgesic, an analeptic, an antiallergic, an antiarrhythmic, an anti-arteriosclerotic, an antibiotic, an antidiabetic, an antifibrinolytic, an anticonvulsive, an angiogenesis inhibitor, an anticholinergic, an enzyme, a coenzyme or a corresponding inhibitor, an antihistaminic, an antihypertensive, an antihypotensive, an anticoagulant, an antimycotic, an antiseptic, an antiinfective, an antihemorrhagic, a betareceptor and calcium channel antagonist, an antimyasthenic, an antiphlogistic, an antipyretic, an antirheumatic, an antiseptic, a cardiotonic, a chemotherapeutic, a coronary dilatator, a cytostatic, a glucocorticoid, a haemostatic, an immunoglobuline or its fragment, a chemokine, a cytokine, a prodrug of a cytokines, a mitogen, a physiological or pharmacological inhibitor of mitogens, a cell differentiation factor, a cytotoxic agent and prodrugs thereof, a hormone, an immunosuppressant, an immunostimulant, a mineralcorticoid, a morphine antagonist, a muscle relaxant, a narcotic, a vector, a peptide, a (para)sympathicomimetic or (para)sympatholytic, a protein, a cell, a selective estrogen receptor modulator (SERM), a sedating agent, a spasmolytic, a substance that inhibits the resorption of bone, a vasoconstrictor or vasodilatator, a virustatic, and a wound healing substance.

13. The encasement of Clause 12, wherein the biologically active substance is selected from one or more of the group consisting of an androgen or antiandrogen, an anaesthetic or analgesic, an antibiotic, an antiarrhythmic, an anti-arteriosclerotic, an antifibrinolytic, an angiogenesis inhibitor, an anticholinergic, an enzyme, a coenzyme or a corresponding inhibitor, an antihypertensive, an antihypotensive, an anticoagulant, an antimycotic, a betareceptor and calcium channel antagonist, an antiphlogistic, a coronary dilatator, a cytostatic, a glucocorticoid, a haemostatic, an immunoglobuline or its fragment, a chemokine, a cytokine, a prodrug of a cytokines, a mitogen, a physiological or pharmacological inhibitor of mitogens, a cell differentiation factor, a cytotoxic agent and prodrugs thereof, a hormone, an immunosuppressant, a mineralcorticoid, a morphine antagonist, a vector, a peptide, a protein, a cell, a selective estrogen receptor modulator (SERM), a sedating agent, a spasmolytic, a substance that inhibits the resorption of bone, a vasoconstrictor or vasodilatator, a virustatic, and a wound healing substance.

14. The encasement of Clause 13, wherein the biologically active substance is selected from one or more of the group consisting of:
(a) an antimicrobial agent or an antifungal agent (e.g. the antimicrobial agent may be selected from one or more of the group consisting of tobramycin, or more particularly tetracycline and its derivatives (such as minocycline, tigecycline and doxycycline), rifampin, triclosan, chlorhexidine, penicillins, aminoglycides, quinolones, vancomycin, gentamycine, a cephalosporin (e.g. cephalosporin), carbapenems, imipenem, ertapenem, an antimicrobial peptide, cecropin-mellitin, magainin, dermaseptin, cathelicidin, a-defensins, a-protegrins and pharmaceutically acceptable salts thereof (e.g. a combination of rifampin and another antimicrobial agent, such as a combination of rifampin and a tetracycline derivative), the antimicrobial agent may be a combination of rifampin and one or more of the group selected from minocycline, doxycycline, and tigecycline (e.g. rifampin and doxycycline, rifampin and tigecycline or, more particularly, rifampin and minocycline, such as a combination of rifampin and/or minocycline, for example, a combination of rifampin and minocycline, the ratio of rifampin to minocycline is from 1:10 to 10:1 (wt/wt) (e.g. from 2:5 to 5:2 (wt/wt)), the antifungal agent may be selected from one or more of the group consisting of azoles (such as ketoconazole, clotrimazole, miconazole, econazole, itraconazole, fluconazole, bifoconazole, terconazole, butaconazole, tioconazole, oxiconazole, sulconazole, saperconazole, clotrimazole, voriconazole, clotrimazole), allylamines (such as terbinafine), morpholines (such as amorolfine and naftifine), griseofulvin, haloprogin, butenafine, tolnaftate, nystatin, cyclohexamide, ciclopirox, flucytosine, terbinafin, amphotericin B and pharmaceutically acceptable salts thereof;
(b) anti-thrombotic agents such as heparin, heparin derivatives, urokinase, and PPack (dextrophenylalanine proline arginine chloromethylketone);
(c) anti-inflammatory agents such as dexamethasone, prednisolone, corticosterone, budesonide, estrogen, sulfasalazine and mesalamine;
(d) anesthetic agents such as lidocaine, bupivacaine and ropivacaine;
(e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides;
(f) vascular cell growth promoters such as hyaluronic acid, growth factors (Ciliary neurotrophic factor, fibroblast growth factors, hepatocyte growth factor, bone morphogenetic proteins), transcriptional activators, and translational promotors;
(g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
(h) protein kinase and tyrosine kinase inhibitors (e.g., tyrphostins, genistein, quinoxalines);
(i) cytotoxic agents, cytostatic agents and cell proliferation affectors;
(j) vasodilating agents;
(k) agents that interfere with endogenous vasoactive mechanisms;
(l) inhibitors of leukocyte recruitment, such as monoclonal antibodies;
(m) bone morphogenetic proteins, such as cytokines and metabologens;
(n) hormones;
(o) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin;
(p) alpha receptor antagonist (such as doxazosin, Tamsulosin) and beta receptor agonists (such as dobutamine, salmeterol), beta receptor antagonist (such as atenolol, metaprolol, butoxamine), angiotensin-II receptor antagonists (such as losartan, valsartan, irbesartan, candesartan and telmisartan), and antispasmodic drugs (such as oxybutynin chloride, flavoxate, tolterodine, hyoscyamine sulfate, diclomine);
(q) bARKct inhibitors;
(r) phospholamban inhibitors;
(s) Serca 2 gene/protein; and
(t) immune response modifiers including aminoquizolines, for instance, imidazoquinolines such as resiquimod and imiquimod.

15. The encasement of any one of the preceding clauses, wherein the at least one elastic sheet may further comprise holes, optionally wherein the diameter of each of the holes is from 0.1 mm to 5 mm (e.g. from 0.3 mm to 2 mm), optionally:
(i) the shape of the holes are uniform and/or the holes are circular; and/or
(ii) the size of the holes are not uniform; and/or
(iii) the holes on the band are evenly distributed throughout the band, focused in the middle (avoiding seals) or nearer to the seals.

16. The encasement of any one of the preceding clauses, wherein the encasement is selected from the group consisting of a pacemaker encasement, or, more particularly, an orthopedic implant encasement, a dental implant encasement, a simulator/sensory implant encasement, a subcutaneous implant encasement, a monitoring implant (e.g. biosensor chip) encasement, a breast implant encasement, an intra-uterine device encasement, an ear tubes (tympanostomy tube) encasement, and a tubing (e.g. catheters) encasement, where the encasement covers at least part of said implant.

17. The encasement of Clause 16, wherein at least a portion of the encasement is dimensionally smaller than the implant to which it is to be applied to and which portion provides a gripping force when the encasement is applied to said implant.

18. The encasement of any one of the preceding clauses, wherein the at least one sheet of elastic material has an elastic recovery of from 80% to 100% (e.g. from 85% to 100%, from 90% to 100% or from 95% to 100%) following stretching up to 300% (e.g. stretching to 100%) elongation and comprises at least one polymer that is biologically-compatible and resorbable.

19. A method of forming elastic medical implant encasement, comprising
(a) providing at least one sheet of an elastic material that further comprises at least one biologically active substance in at least one region of the sheet; and
(b) forming the at least one sheet into the elastic medical implant encasement.

20. The method of Clause 19, wherein the method comprises:
(A)
(i) providing one sheet of an elastic material that further comprises at least one biologically active substance in at least one region of the sheet;
(ii) folding at least a part of the sheet onto itself to form an edge; and
(iii) sealing at least part of the edge to form the elastic medical implant encasement; and/or

(B)
(i) providing at least two sheets of elastic material where at least one of the sheets further comprises at least one biologically active substance in at least one region of said sheet;
(ii) overlapping the at least two sheets in at least one area to form an overlapping area; and
(iii) sealing at least part of the overlapped area to form the elastic medical implant encasement; and/or

(C)
providing at least one seamless tubular structure of elastic material having at least one biologically active substance in at least one region.

21. The method of Clause 19 or Clause 20, wherein forming and/or sealing is accomplished using one or more of the methods selected from the group consisting of heat fusion, chemical fusion, and adhesives.

22. A medical implant at least partly covered with an elastic medical implant encasement according to any one of Clauses 1 to 18.

23. The medical implant of Clause 22, wherein the medical implant is selected from the group consisting of a pacemaker, or, more particularly, an orthopedic implant, a dental implant, a simulator/sensory implant, a subcutaneous implant, a monitoring implant (e.g. biosensor chip), a breast implant, an intra-uterine device, an ear tube (tympanostomy tube), and a tubing (e.g. catheters).

### BRIEF DESCRIPTION OF DRAWINGS

The features of the preferred embodiments will be described, with reference to the following drawings where like elements are labeled similarly, and in which:

FIG. 1 depicts a schematic illustration of the top view of a bioresorbable encasement designed according to an embodiment of the invention and a medical implant in the form of a bone plate that is insertable into the encasement;

FIG. 2 depicts schematic illustrations of an elastic bioresorbable encasement according to another embodiment of the present invention. FIG. 2A depicts a side view of the encasement, while FIG. 2B depicts the bottom view of the encasement of FIG. 2A.

FIG. 3 is a side view of the embodiment of FIG. 2 and a medical implant in the form of a bone plate that is insertable into the encasement;

FIG. 4 is a side view of one embodiment of an odd-shaped elastic bioresorbable encasement according to principles of the present invention and an odd-shaped medical implant in the form of a hip implant that is insertable into the encasement;

FIG. 5 is a side view of one embodiment of a tubular elastic bioresorbable encasement according to principles of the present invention and an odd-shaped medical implant in the form of an orthopedic screw that is insertable into the encasement;

FIG. 6 is a graph plotting force against strain (or elongation) percentage for a representative film suitable for use in the current invention, as well as said film's elastic recovery once the strain is released.

FIGS. 7-1 to 7-8 depict examples of layered designs according to embodiments of the current invention. The numbering within each of FIGS. 7-1 to 7-8 refers to the preparative example for each layer. Layer 7-C(V) in FIG. 7-8 refers to the use of example 7-C to prepare a layer, but where the active agents are replaced by vancomycin.

FIG. 8 depicts the cumulative release profile of minocycline (8-1) and rifampin (8-2) in the exemplified embodiments of the invention.

FIG. 9 depicts the cumulative release profile of minocycline and rifampin in a single film according to an embodiment of the current invention.

FIG. 10 depicts the cumulative release profile of vancomycin in a single film according to an embodiment of the current invention.

### DESCRIPTION

The invention is generally directed to an elastic biologically-compatible encasement which serves the purpose of carrying biologically active agents for site specific features for use with medical implants. The device may comprise more than one biologically active agents and one or more layers of biodegrable polymer films. The polymer films may be constructed as single layer or layer-by-layer structure. The biologically active agents may be incorporated into one or all or some layers of biodegradable polymer films. The biologically active agents may be released locally to the surrounding tissue over time.

The current invention provides a surprisingly effective alternative method to those described hereionbefore, such as directly coating an implant with a polymer and/or a biologically active material. Thus, there is provided an elastic medical implant encasement, comprising:
at least one sheet of elastic material configured to form an encasement for at least part of a medical implant; and
at least one biologically active substance in at least one region of the at least one sheet of elastic material, wherein
the at least one sheet of elastic material comprises at least one polymer that is biologically-compatible and resorbable and has an elastic recovery of from 80% to 100% following stretching, or can stretch from its original size to an expanded size and return to its original size or to a size no greater than the expanded size minus 80% of the difference between expanded size and original size.

For example, the encasement or film can stretch from its original size to an expanded size and return to its original size or to a size no greater than the expanded size minus 90% of the difference between expanded size and original size.

Alternatively or additionally, the at least one sheet of elastic material has an elastic recovery of from 80% to 100% (e.g. from 85% to 100%, from 90% to 100% or from 95% to 100%) following stretching up to 300% (e.g. stretching to 100%) elongation.

Encasements of the current invention are made of an elastic bioabsorbable polymer that can be fitted snugly around at least part of a medical implant. The encasement acts as a carrier of one or more biologically-active agents (such as, but not limited to, antibiotics, or more particularly, growth agents etc) for specific purposes. The encasement is proportioned and made of an elastic material so that it conform to the shape and size of the portion of the implant to which it is applied, thereby providing a snug fit around the portion of the implant it is applied to. Given this, the impant can largely retain its original size, shape and function, without having its effectiveness compromised.

As will be appreciated, the proportions of the elastic encasement are intended to be smaller than that of the portion of the medical implant it is intended to be affixed to. Given this, when the elastic encasement is applied to the medical implant, it will provide a grip-force between the elastic encasement and the medical implant, such that the encasement will not drop off or move during normal use and during implantation of the medical implant (even if the encasement-covered implant is subjected to shear forces during implantation). As the encasement may be made of a resorbable material, the encasement can dissolve in the human or animal body after a given period of time, such that only the implant itself is left behind, if the implant is non-biodegradable. The elastic medical implant encasement described herein can deliver a site specific function with minimal distortion to the medial implant's shape and way of use, while allowing customisation of the biologically active agents provided with the implant to better suit the needs of the subject undergoing treatment. The snug fit and grip force between the article and implant are due to the elasticity of the polymer(s) used.

Current commercially available implant encasements for site-specific functions lack any grip force and so need to be larger than the implant to which it is applied. This leads to the issues discussed hereinbefore, such as not being able to use an encasement due to lack of space to fit the encasement into the desired implantation site, or movement of the implant within the encasement, leading to improper coverage of the implant, leading to infections. In contrast, the ability to retain the medical implant in a fixed position relative to the encasement before, during and after successful implantation is due to the grip force generated by the elasticity of the encasement material. This elasticity enables encasements of the current invention to be designed to have any shape or size that can retain whole or part of a medical implant firmly, provided that at least part of the design can mechanically produce a grip force on the implant. This opens up the scope of encasement design tremendously compared to current technologies. For example, as encasements used currently have to be provided in a form larger than the implant, this results in space between the encasement and the implant, leading to a need for a larger implantation site to accommodate the extra space, which may not always be possible or desirable. The encasement of the invention alleviates this need for extra space and thereby eliminates (or at least substantially reduces) the need for implantation site enlargement.

Elastic encasements as described herein can conform exactly to whole or part of the shape and design of the implant, which current encasements cannot do. This is highly advantageous, as it enables the retention of the ergonomic design and function of the original implant, which may be crucial to its use. For example, current encasements are not suitable for use with implants where shape affects the functionality of the implant, such implants include, but are not limited to, hip replacement implants or dental implant screws. The firm gripping and ability to conform to the shape of an implant means that the encasements described herein can be used for difficult implantations, such as the insertion of the stem implant in an artificial hip replacement, or the insertion of screws used in dental or orthopedic implants, which implantation procedures generate a high degree of shear force or other mechanical destructive force, and the implantation site has limited space. Thus, the current encasements further widen the potential uses of encasements in medical implantations.

Without wishing to be bound by theory, the ability to conform to the shape of a medical implant may also translate to a better efficiency of the agents being applied. For example, if the article in the invention is preloaded with antibiotics for purpose of anti-infection, the snug fit encasement on the surface of the implant could better prevent bio-film from forming on the surface of the implant. For another example, if the article in invention is preloaded with osteointegration agents for bone implants, the snug fit encasement can help to better promote bone growth near the surface of the implant where the encasement is.

The term “encasement” as used herein relates to an object that partly or wholly covers a medial device. In particular, a part or the whole of the encasement is intended to be fixed onto part or the whole of a medical device and be held in place by a grip force provided by one or more anchoring portions of the encasement. The anchoring portions of the encasement provide the grip force discussed above by being smaller in dimension than the implant to which they are applied and being elastic. As such, the anchoring portions have to be expanded to a size bigger than the medical implant portion to which they are attached in order to enable them to be fitted into place, but then the anchoring portions elastically recover towards their original size, thereby providing a grip force effect on the surface of the medical implant to which they are applied.

The encasement may be in any suitable form that results in at least a portion of the medical implant being covered by the encasement. Suitable forms that may be mentioned herein include, but are not limited to a mesh, pouch, bag, envelop, sleeve, pocket or receptacle, all of which may optionally include apertures, bands, or designs that enable the encasement to grip onto at least a portion of the implant surface.

When used herein “anchoring portions” may refer to the whole of an encasement (e.g. a pouch, where the elasticity of the entire pouch provides the grip force on the surface of an implant to which is it applied and hence the entire pouch acts as an anchoring portion) or to part of an encasement (e.g. elastic loops affixed to the main body of the encasement).

The term “at least one elastic sheet” is intended to cover the situation where there is only one elastic sheet, or more than one elastic sheet as the case may be (e.g. from two to twenty, from three to fifteen, from four to ten etc). The term “sheet” when used herein is not intended to refer solely to flat objects that may be folded and sealed to create more complex objects (e.g. an envelope), but is also intended to cover seamless objects, such as a tube-shaped sheet, which may have been formed by extrusion in a single piece and which is seamless. It will be appreciated that more than one sheet may be used in conjunction to provide the encasements and this will be discussed in more detail hereinbelow.

Thus, in certain embodiments of the invention, thin layers of elastic bioabsobable material may be manufactured into different designs of implant encasement to encasement the medical devices prior to implantation. The different designs of encasement is to optimised the evenness of the material on the implants. The article is designed to be smaller than the implant, at least at one part, for the elasticity to create a force to grip firmly on the implant. Different agents, such as antibiotics for treatment of infection and osteoconductive agent for bone growth, can be carried by the article. The agents can be carried via layered implementations. The article can have layer or layers of the same agent or different layers of different agents. Further discussion of the agent are provided hereinbelow.

Examples of suitable encasement forms include, but are not limited to:
(a) at least one sheet of elastic material with two or more anchoring points formed via folding onto itself or with at least one additional sheet of elastic material, where at least one of the elastic material sheets carries at least one biologically active substance in at least one region;
(b) at least one sheet of elastic biologically-compatible, resorbable, material folded onto itself to form a single large anchoring surface, where the at least one elastic material sheet carries at least one biologically active substance in at least one region;
(c) at least two sheets of elastic material sealed at overlapping areas to form one or more anchoring points or surfaces, where at least one of the elastic material sheets carries at least one biologically active substance in at least one region; or
(d) the encasement comprises a seamless tubular structure formed from at least one sheet of elastic material, where the at least one elastic material sheet carries at least one biologically active substance in at least one region.

In embodiments of the invention that may be mentioned herein, each of the at least one sheet(s) of elastic material may have a thickness of from 0.01μηι to 1000μηι.

In embodiments of the invention that may be mentioned herein, the at least one elastic sheet may further comprise holes. For example, the diameter of each of the holes may be from 0.1 mm to 5 mm (e.g. from 0.3 mm to 2 mm). For the avoidance of doubt, unless specified herein, the holes may have any shape and may be uniform or irregular in shape, as well as size. In particular examples that may be mentioned herein one or more of the following may apply:
(i) the shape of the holes are uniform and/or the holes are circular;
(ii) the size of the holes are not uniform; and
(iii) the holes on the band are evenly distributed throughout the band, focused in the middle (avoiding seals) or nearer to the seals.

For example, in certain embodiments, the shape of the holes may be entirely uniform or entirely irregular across the entire encasement. However, in certain cases, the encasement may have one or more regions that have holes with a uniform shape and one or more regions where the holes are irregular. Other arrangements may be envisaged within the scope of the possible combinations of the above-mentioned features.

The terms “medical implant” and “implantable medical device” refers to any medical device that can be implanted transdermally, or any in-dwelling medical device that includes a transdermal component. Examples of medical implants that may be mentioned herein include, but are not limited to, orthopedic implants, dental implants, simulator/sensory implants, subcutaneous implants, monitoring implants (e.g. biosensor chips), breast implants, intra-uterine devices, ear tubes (tympanostomy tubes), implantable tubing (e.g. catheters), arterio venous shunts, left ventricular assist devices, tissue expanders, gastric lap bands, and intrathecal infusion pumps.

Examples of orthopedic implants that may be mentioned herein includes, but is not limited to, hip replacements, knee replacements, shoulder replacement, elbow replacements, ankle replacements, neck/spine artificial discs, screws (e.g. neck/spine screws), pins, plates, and rods (e.g. neck/spine rods).

Examples of dental implants that may be mentioned herein includes, but is not limited to, endosteal implants and subperiosteal implants (e.g. mandibular endoprothesis/plate, and dental implant abutments)

Examples of simulator/sensory implants that may be mentioned herein includes, but is not limited to, brain (or neural) implants (e.g. implantable neurostimulator (INS), deep brain stimulators), spinal cord stimulators, gastric electrical stimulators, sacral nerve stimulators, vagus nerve stimulators, and Cochlear implants.

It will be appreciate that the encasements may be suited for a single particular purpose or may be suitable for use with more than one implant, depending on the size and dimensions of the encasement in question. For the avoidance of doubt, the encasements may be one or more of a pacemaker encasement, or, more particularly, an orthopedic implant encasement, a dental implant encasement, a simulator/sensory implant encasement, a subcutaneous implant encasement, a monitoring implant (e.g. biosensor chip) encasement, a breast implant encasement, an intra-uterine device encasement, an ear tubes (tympanostomy tube) encasement, and a tubing (e.g. catheters) encasement, where the encasement covers at least part of said implant. Other encasements may be derived by analogy to the listing of medical implants provided herein. In particular embodiments of the invention, the encasement is not a CIED encasement.

The term “elastic recovery” as used in the present invention refers to the ability of the whole or part of the encasement to be reversibly extended or plastically deformed in at least one direction, preferably in two directions, upon application of a force and recover towards its original size once the force is removed.

The encasement is stretchable to at least 1.1 times (e.g. from 1.2 times to 10 times) to allow for insertion of the implant into the encasement, and can recover to more than 80% to securely hold the implant within the encasement and prevent them separating during implantation. A construction of the encasement that may be mentioned herein comprises at least one film, which itself comprises at least one polymer layer and at least one antimicrobial agent; and at least one opening and numerous holes on the surface.

The elastic sheets used herein may be stretched up to 10 times its original size in any direction (e.g. from 1.1 times to 4 times its original size) and may then recover at least to 80%, such as at least 90% of its original size following release of the stretch. For example, when stretching a film to size B (a difference of size C) from size A results in the film returning to a maximum size of B−(0.8×C) following stretching and release, where C is B−A, such as a maximum size of B−(0.9×C). That is, if one stretches a film from 0.1 cm to 0.1 1 cm (difference of 0.01 cm), the resulting film will have maximum size of 0.1 1−(0.8×0.01)=0.102 cm if the film recovers at least to 80% of its original size or will have a maximum size of 0.101 cm if the film recovers to at least 90% of its original size following stretching. It will be appreciated that the film may recover to its original size or almost to its original size. Additionally or alternatively, the elastic sheet(s) used in the current invention may have an elastic recovery of from 80% to 100% following stretching to 100% of its original length. For example, if an elastic sheet measuring 1×1 cm is stretched in at least one direction to a size of 2 cm, the sheet will recover to at least 1.2 cm (e.g. from 1.2 cm to 1 cm) in the direction(s) stretched. In particular embodiments of the invention that may be mentioned herein the elastic recovery exhibited by the one or more elastic sheets may be from 85% to 100%, from 90% to 100% or from 95% to 100% following stretching to 100% of its original length. It will be appreciated that the elasticity of the sheet is not only dependent on the composition of the polymer material itself but also on the structure imparted to said material during its processing.

In embodiments of the invention, at least a portion of the encasement is dimensionally smaller than the implant to which it is to be applied to and which portion provides a gripping force when the encasement is applied to said implant. This is due to the gripping force provided by the elastic recovery of the at least one elastic sheet used in the encasement.

The term “resorbable” or “bioresorbable” as used herein refers to a polymeric material that can be dissolved or degraded when in contact with tissue and/or fluids in the body of a subject by e.g. enzymatic or chemical means. Resorbable polymers that may be mentioned herein include, but are not limited to poly(lactide-co-caprolactone), poly(DL-lactide-co-caprolactone) (DL-PLCL), Poly(L-lactide-co-caprolactone) (PLLCL)polycaprolactone (PCL), polyglycolide (PGA), poly(L-lactic acid) (PLLA), poly(glycolide-co-caprolactone) (PGCL) copolymer, poly(D,L-lactic acid), poly(L-lactide-co-D, L-lactide) (PLDLLA), poly(L-lactide-co-glycolide) (PLGA), poly(D,L-lactide-co-glycolide), poly (D-lactide) (PDLA), poly(trimethylene carbonate) (PTMC), poly(lactide-co-trimethylene carbonate) (PLTMC), poly(gycolide-trimethylene carbonate), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives and copolymers thereof (e.g. the bioresorbable polymer of the at least one polymer layer may be selected from one or more of the group consisting of poly(DL-lactide-co-caprolactone) (DL-PLCL), or more particularly, polycaprolactone (PCL), polyglycolide (PGA), poly(L-lactic acid) (PLA), polydioxanone (PDO), poly(4-hydroxy butyrate) (PHB), polyhydroxyalkanoates (PHA), PEG and its derivatives, and their copolymers (such as selected from one or more of the group consisting of poly(DL-lactide-co-caprolactone) (DL-PLCL), or more particularly, poly(L-lactide-co-caprolactone) (PLLCL), poly(glycolide-co-caprolactone) (PGCL) copolymer, or more preferably, polycaprolactone (PCL), polyglycolide (PGA), poly(L-lactic acid) (PLA), PEG and its derivatives and their copolymers. Particular polymers that may be mentioned include polycaprolactone (PCL), poly(DL-lactide-co-caprolactone) (DL-PLCL), poly(glycolide-co-caprolactone) (PGCL), poly(lactide-co-caprolactone) (PLCL) and its derivatives and their copolymers)).

For example, the resorbable polymers may be selected from poly(lactide-co-caprolactone), poly(DL-lactide-co-caprolactone) (DL-PLCL), poly(L-lactide-co-caprolactone) (PLLCL), polycaprolactone (PCL), polyglycolide (PGA), poly(L-lactic acid) (PLLA), poly(glycolide-co-caprolactone) (PGCL) copolymer, poly(D,L-lactic acid), poly(L-lactide-co-D, L-lactide) (PLDLLA), poly(L-lactide-co-glycolide) (PLGA), poly(D,L-lactide-co-glycolide), poly (D-lactide) (PDLA), poly(trimethylene carbonate) (PTMC), poly(lactide-co-trimethylene carbonate) (PLTMC), poly(gycolide-trimethylene carbonate), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone-co-butylacrylate, a copolymer of polyhydroxybutyrate, a copolymer of maleic anhydride, a copolymer of poly(trimethylene carbonate), polyethylene glycol, hydroxypropylmethylcellulose and cellulose derivatives, polysaccharides, such as hyaluronic acid, chitosan, starch, proteins such as gelatin, collagen or PEG derivatives, and blends thereof.

In particular embodiments of the invention that may be mentioned herein, the elastic sheets may be made from one or more polymer sheets, where each sheet may be made from:
a) poly(lactide-co-caprolactone) (PLCL) (e.g. having a PLA to PCL ratio of from 90:10 to 60:40) or its derivatives and copolymers thereof; and/or
b) poly(DL-lactide-co-caprolactone) (DL-PLCL) (e.g. having a DL-PLA to PCL ratio of from 90:10 to 50:50) or its derivatives and copolymers thereof; and/or c) poly(glycolide-co-caprolactone) (PGCL) (e.g. having a PGA to PCL ratio of from 90:10 to 10:90) or its derivatives and copolymers thereof; and/or d) a blend of PLCL or DL-PLCL or PGCL with a releasing agent selected from one or more of the group selected from polysorbate 20, polysorbate 40, polysorbate 60, polysorbate 80, or polyethyleneglycol having a molecular weight of 200 to 2000 Daltons in a w wt ratio of PLCL or DL-PLCL or PGCL to releasing agent of from 25:1 to 1:9.

In particular embodiments of the invention that may be mentioned herein, the number average molecular weight of the polymer may be 10,000-2,000,000 Daltons, preferably 50,000-1,500,000.

Unless otherwise specified herein, polymers in the form of copolymers may be random copolymers, alternating copolymers with regular alternating A and B units, periodic copolymers with A and B units arranged in a repeating sequence (e.g. (A-B-A-B-B-A-A-A-A-B-B-B)n), random copolymers, block copolymers comprise two or more homopolymer subunits linked by covalent bonds. In particular embodiments of the invention, copolymers may be random block copolymers.

References herein (in any aspect or embodiment of the invention) to “biologically active substance” and/or “biological agent” includes references to such substances/agents per se, as well as to pharmaceutically acceptable salts or solvates of such substances/agents.

Pharmaceutically acceptable salts that may be mentioned include acid addition salts and base addition salts. Such salts may be formed by conventional means, for example by reaction of a free acid or a free base form of a substance/agent with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a substance/agent in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1 S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), a-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by the biologically active substances/biological agents described herein are any solvates of the substances/agents and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, Ind., USA, 1999, ISBN 0-967-06710-3.

Biologically active substances/biological agents described herein are intended to be administered as part of the encasement, when the encasement is fitted to a medical implant. As such, the biologically active substances/biological agents described herein are generally administered as part of the encasement and may be coated on a portion of a surface of the encasement (i.e. coated on a surface of part of one of the at least one sheet of elastic material) and/or it may be encapsulated within the at least one sheet of elastic material.

It will be appreciated that the at least one sheet of elastic material may have only one sheet or it may have more than one sheet, for example from two to ten sheets of elastic material. In embodiments of the invention where there are two or more sheets the biologically active substance may be encapsulated within one or more (e.g one) of the two or more sheets of elastic material and/or is coated on the surface of one or more (e.g one) of the two or more sheets of elastic material, optionally wherein the coated surface is not an outer surface of the two or more sheets of elastic material (and so is effectively encapsulated between at least two sheets of elastic material). For the avoidance of doubt, each sheet of elastic material may be made from the same polymeric material as the other sheets, or each sheet may be made using different materials, or any combination in-between these extremes.

It will be appreciated that the biologically active substances/biological agents described herein may be provided in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995). A brief review of methods of drug delivery may also be found in e.g. Langer, Science (1990) 249, 1527.

Otherwise, the preparation of suitable formulations for use in the current invention may be achieved routinely by the skilled person using routine techniques and/or in accordance with standard and/or accepted pharmaceutical practice.

The amount of the biologically active substances/biological agents described herein in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, the implant may contain from 0.001 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment and from 1 to 99.9% w/w of the polymeric material.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease. In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The biologically active substances/biological agents that may be mentioned herein may be adrenocorticostatic, a β-adrenolytic, an androgen or antiandrogen, an antianemic, an antiparasitic, an anabolic, an anaesthetic or analgesic, an analeptic, an antiallergic, an antiarrhythmic, an anti-arteriosclerotic, an antibiotic, an antidiabetic, an antifibrinolytic, an anticonvulsive, an angiogenesis inhibitor, an anticholinergic, an enzyme, a coenzyme or a corresponding inhibitor, an antihistaminic, an antihypertensive, an antihypotensive, an anticoagulant, an antimycotic, an antiseptic, an antiinfective, an antihemorrhagic, a betareceptor and calcium channel antagonist, an antimyasthenic, an antiphlogistic, an antipyretic, an antirheumatic, an antiseptic, a cardiotonic, a chemotherapeutic, a coronary dilatator, a cytostatic, a glucocorticoid, a haemostatic, an immunoglobuline or its fragment, a chemokine, a cytokine, a prodrug of a cytokines, a mitogen, a physiological or pharmacological inhibitor of mitogens, a cell differentiation factor, a cytotoxic agent and prodrugs thereof, a hormone, an immunosuppressant, an immunostimulant, a mineralcorticoid, a morphine antagonist, a muscle relaxant, a narcotic, a vector, a peptide, a (para)sympathicomimetic or (para)sympatholytic, a protein, a cell, a selective estrogen receptor modulator (SERM), a sedating agent, a spasmolytic, a substance that inhibits the resorption of bone, a vasoconstrictor or vasodilatator, a virustatic, and a wound healing substance. For example, the biologically active substance is selected from one or more of the group consisting of an androgen or antiandrogen, an anaesthetic or analgesic, an antibiotic, an antiarrhythmic, an anti-arteriosclerotic, an antifibrinolytic, an angiogenesis inhibitor, an anticholinergic, an enzyme, a coenzyme or a corresponding inhibitor, an antihypertensive, an antihypotensive, an anticoagulant, an antimycotic, a betareceptor and calcium channel antagonist, an antiphlogistic, a coronary dilatator, a cytostatic, a glucocorticoid, a haemostatic, an immunoglobuline or its fragment, a chemokine, a cytokine, a prodrug of a cytokines, a mitogen, a physiological or pharmacological inhibitor of mitogens, a cell differentiation factor, a cytotoxic agent and prodrugs thereof, a hormone, an immunosuppressant, a mineralcorticoid, a morphine antagonist, a vector, a peptide, a protein, a cell, a selective estrogen receptor modulator (SERM), a sedating agent, a spasmolytic, a substance that inhibits the resorption of bone, a vasoconstrictor or vasodilatator, a virustatic, and a wound healing substance.

In particular embodiments that may be mentioned herein, the biologically active substance may be selected from one or more of the group consisting of an adrenocorticostatic, a β-adrenolytic, an androgen or antiandrogen, an antianemic, an anaesthetic or analgesic, an analeptic, an antiarrhythmic, an anti-arteriosclerotic, an antidiabetic, an antifibrinolytic, an anticonvulsive, an angiogenesis inhibitor, an anticholinergic, an antihypertensive, an antihypotensive, an anticoagulant, an antimycotic, a betareceptor and calcium channel antagonist, an antimyasthenic, an antiphlogistic, an antirheumatic, a cardiotonic, a coronary dilatator, a cytostatic, a glucocorticoid, a haemostatic, a cell differentiation factor, a cytotoxic agent and prodrugs thereof, a hormone, an immunosuppressant, an immunostimulant, a mineralcorticoid, a morphine antagonist, a muscle relaxant, a narcotic, a (para)sympathicomimetic or (para)sympatholytic, a selective estrogen receptor modulator (SERM), a sedating agent, a spasmolytic, a substance that inhibits the resorption of bone, a vasoconstrictor or vasodilatator, and a wound healing substance.

When used herein, the term “analgesic” means any drug that provides an analgesic effect or any drug that provides a blockage of nociceptive pain and/or neuropathic pain. Analgesics that may be mentioned herein include, but are not limited to, buprenorphine, nalbuphine, benzocaine, dyclonine HCl, phenol, aspirin, phenacetin, acetaminophen, potassium nitrate, and pharmaceutically acceptable salts thereof, and mixtures thereof.

Antineoplastic agents that may be mentioned herein include, but are not limited to, doxorubicin, vinblastine, vincristine, 5-fluorouracil (5-FU), daunorubicin, epirubicin, mitoxanthrone, and cyclophosphamide or combinations thereof.

Bisphosphonates that may be mentioned herein include, but are not limited to, etidronate, clodronate, tiludronate, neridronate, olpadronate, alendronate, ibandronate, risedronate and zoledronate, or combinations thereof.

Examples of more particular biological agents that may be used herein include, but are not limited to:
(a) an antimicrobial agent or an antifungal agent (e.g. the antimicrobial agent may be selected from one or more of the group consisting of tobramycin, or more particularly, tetracycline and its derivatives (such as minocycline, tigecycline and doxycycline), rifampin, triclosan, chlorhexidine, penicillins, aminoglycides, quinolones, vancomycin, gentamycine, a cephalosporin (e.g. cephalosporin), carbapenems, imipenem, ertapenem, an antimicrobial peptide, cecropin-mellitin, magainin, dermaseptin, cathelicidin, a-defensins, a-protegrins and pharmaceutically acceptable salts thereof (e.g. a combination of rifampin and another antimicrobial agent, such as a combination of rifampin and a tetracycline derivative), the antimicrobial agent may be a combination of rifampin and one or more of the group selected from minocycline, doxycycline, and tigecycline (e.g. rifampin and doxycycline, rifampin and tigecycline or, more particularly, rifampin and minocycline, such as a combination of rifampin and/or minocycline, for example, a combination of rifampin and minocycline, the ratio of rifampin to minocycline is from 1:10 to 10:1 (wt/wt) (e.g. from 2:5 to 5:2 (wt/wt)), the antifungal agent may be selected from one or more of the group consisting of azoles (such as ketoconazole, clotrimazole, miconazole, econazole, itraconazole, fluconazole, bifoconazole, terconazole, butaconazole, tioconazole, oxiconazole, sulconazole, saperconazole, clotrimazole, voriconazole, clotrimazole), allylamines (such as terbinafine), morpholines (such as amorolfine and naftifine), griseofulvin, haloprogin, butenafine, tolnaftate, nystatin, cyclohexamide, ciclopirox, flucytosine, terbinafin, amphotericin B and pharmaceutically acceptable salts thereof;
(b) anti-thrombotic agents such as heparin, heparin derivatives, urokinase, and PPack (dextrophenylalanine proline arginine chloromethylketone);
(c) anti-inflammatory agents such as dexamethasone, prednisolone, corticosterone, budesonide, estrogen, sulfasalazine and mesalamine;
(d) anesthetic agents such as lidocaine, bupivacaine and ropivacaine;
(e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides;
(f) vascular cell growth promoters such as hyaluronic acid, growth factors (Ciliary neurotrophic factor, fibroblast growth factors, hepatocyte growth factor, bone morphogenetic proteins), transcriptional activators, and translational promotors;
(g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
(h) protein kinase and tyrosine kinase inhibitors (e.g., tyrphostins, genistein, quinoxalines); (i) cytotoxic agents, cytostatic agents and cell proliferation affectors;
(j) vasodilating agents;
(k) agents that interfere with endogenous vasoactive mechanisms;
(l) inhibitors of leukocyte recruitment, such as monoclonal antibodies;
(m) bone morphogenetic proteins, such as cytokines and metabologens;
(n) hormones;
(o) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin;
(p) alpha receptor antagonist (such as doxazosin, Tamsulosin) and beta receptor agonists (such as dobutamine, salmeterol), beta receptor antagonist (such as atenolol, metaprolol, butoxamine), angiotensin-II receptor antagonists (such as losartan, valsartan, irbesartan, candesartan and telmisartan), and antispasmodic drugs (such as oxybutynin chloride, flavoxate, tolterodine, hyoscyamine sulfate, diclomine),
(q) bARKct inhibitors;
(r) phospholamban inhibitors;
(s) Serca 2 gene/protein; and
(t) immune response modifiers including aminoquizolines, for instance, imidazoquinolines such as resiquimod and imiquimod.

In yet more particular embodiments of the invention, the biological agents used herein include may be selected from (a) to (g) of the list immediate above.

In certain embodiments that may be mentioned herein, the biologically active substances/biological agents are not antibiotics (e.g. the biologically active substances/biological agents are not antibiotics and the encasement is not a CI ED encasement).

It will be appreciated that any type of drug or biological agent may be carried by the encasement and the invention is not limited by the type used, unless otherwise specified in embodiments of the invention described herein. It should be noted that any reference herein to a “drug” is broadly defined as any medically related biological agent that may beneficially be incorporated into the encasement for dispensing to the implantation site of a medical implant.

As used herein, the term “peptide” includes one or more peptides, peptide derivatives, or combinations thereof. Thus, the terms “peptide”, “peptides”, and “derivatives of peptides” are used interchangeably throughout. “Peptide” refers to both naturally occurring peptides and synthesized peptides, including naturally or nonnaturally occurring amino acids. Peptide derivatives are created by chemically modifying a side chain or a free amino or carboxyterminus of a natural or nonnaturally occurring amino acid. This chemical modification includes the addition of further chemical moieties as well as the modification of functional groups in side chains of the amino acids. A peptide is a polymer of between 3 and 50 amino acids, preferably having more than 3, 5, 10, 15, 20, 30, 40 amino acids. The term “protein” includes one or more proteins, protein derivatives, or combinations thereof and is differentiated from the term “peptide” in that it refers to polymers comprising amino acids chains of more than 50 amino acids.

As used herein, “growth factors” are chemicals that regulate cellular metabolic processes, including but not limited to differentiation, proliferation, synthesis of various cellular products, and other metabolic activities. Growth factors may include several families of chemicals, including but not limited to cytokines, eicosanoids, and differentiation factors.

The present invention provides a “one-size fits all” solution to the foregoing coated-implant problem. Instead of creating an inventory of numerous different coated implants, an encasement with biological agent(s) is alternatively provided that preferably can fit a range of different conventional uncoated/coated implants. According to one aspect of the invention, therefore, the elastic encasement is configured to advantageously accommodate a wide variety of implant types, shapes, and sizes. In the preferred embodiment, the elastic encasement has at least a part of it stretched to fit onto the implant. The stretch will produce a corresponding grip force due to its elasticity.

According to another aspect of the invention, the encasement is implanted into a patient and the active agent(s) is/are dispensed from the encasement in vivo over time to tissue surrounding the implantation site. In one embodiment, the duration and dosage of the agent delivered to the patient from the encasement may be controlled by such factors as the choice of encasement material used, construction of the encasement, and type and form of agent or combination of agents and/or agents' delivery systems impregnated into the encasement as further described herein. The duration of release for different agents can be either same or different. The release of agents can be timed to be independent or simultaneously.

In one embodiment, the encasement can be in the form of pouch, envelope or sleeve where the encasement surround majority of the implant. This provides large area of grip on the implant. In this aspect, it is likely the encasement is smaller than the implant although the embodiment can have only partial of the encasement smaller than the implant.

In another embodiment, the encasement can be design to surround only the implant partially. This can be a film with anchorage points where either the film is stretched over the implant or the anchorage points are stretched over the implants or both. In this aspect, it is likely only part of the encasement is smaller than the implant although the embodiment can have the encasement smaller than the implant.

In another embodiment, the encasement can be designed in accordance to the shape of the implant and is not limited to the regular shapes like, for example, envelope design for pacemaker or elongated design for plates.

Another possible embodiment of a encasement generally includes at least one sheet made of a biologically compatible material and at least one biological active agent impregnated into the encasement.

Another possible embodiment of a encasement includes multiple agents are carried by the biologically compatible material. The agents are carried in different layers and can be arranged in any order or with symmetrical order with reference to the biologically compatible material.

The biologically active agent(s) dispensing rates can be manipulated from days to months. The polymer chemistry and type of polymer used provide a wide range of possible drug delivery kinetics and polymer resorption times. In addition, resorption times and drug delivery rates can be manipulated by the thickness of sheets used to construct the polymer encasement and the addition of releasing agents. Other techniques may be employed for controlling the delivery rate and duration of delivery for drugs or biological agents from the encasement. For example, in certain embodiments of the encasement and/or film of the invention:
(a) the film may have at least two polymer layers. For example, the film may have from two to ten polymer layers (e.g. from two to nine polymer layers, such as from three to seven polymer layers);
(b) at least one of the polymer layers may further comprise a releasing agent that is composed of one or more biocompatible hydrophilic small molecules with a hydrophobic-lipophilic balance of greater than 6 (e.g. the releasing agent is selected from one or more of the group consisting of sorbitol, xylitol, glycerin, mannitol, polyethylene glycol (PEG) having a number average molecular weight of from 200 to 2000, polysorbate and urea (e.g. selected from one or more of polysorbate 40, or more particularly, polysorbate 20, polysorbate 60 and polysorbate 80));
(c) the at least one biologically active agent may be miscible with the bioresorbable polymer of each polymer layer in which it is present;
(d) in at least one layer of the polymer film, the at least one biologically active agent may be homogeneously distributed within at least one of the polymer layers in
which it is present (e.g. when the at least one biologically active agent is distributed within a polymer layer, it is homogeneously distributed within said polymer layer); (e) when the film has at least two polymer layers, the at least one biologically active agent is distributed within at least two of the polymer layers;
(f) when the film has at least two polymer layers, the at least one biologically active agent forms a separate layer sandwiched between the two polymer layers; (g) in at least one layer of the polymer film, the at least one biologically active may be present in an amount of 0.1 wt to 99 wt %, such as from 0.1 wt % to 95 wt % of said polymer layer (e.g. from 0.1 wt % to 90 wt % or from 0.1 wt % to 80 wt %, such as from 0.1 wt % to 60 wt %), for example, in at least one layer of the polymer film, the at least one biologically active agent may be present in an amount of from 0.1 wt % to 30 wt % (e.g. from 1 wt % to 25 wt %) of said polymer layer, optionally wherein said polymer layer is solvent cast and/or in the at least one layer of the polymer film, the at least one biologically active agent may be present in an amount of from 10 wt % to 95 wt % (e.g. from 10 wt % to 60 wt %, or from 30 wt % to 95 wt %, such as from 40 wt % to 80 wt %) of said polymer layer, optionally wherein said polymer layer was spray coated onto a substrate.

In one embodiment, the encasement may contain a plurality of apertures, which in one embodiment may be round perforations or holes. In another embodiment, the apertures shapes could be irregular, the dimensions could be varying. In the preferred embodiment the (aperture area/total area) ratio could be from 0% to 95%.

In one embodiment, the encasement can be form using a single sheet with the anchorage formed by folding at least a part of the sheet onto itself and sealing at least part of the edge to fix the fold.

In another embodiment, the encasement can be form using a multiple sheets with the anchorage formed by overlapping at least 2 different sheets at at least one area and sealing at least part of the overlapped area.

In another embodiment, the encasement can be one seamless tubular structure.

In another embodiment, the encasement can be a combination of seamless tubular structure with sheet(s).

In order that the invention may be understood, preferred embodiments which are given by way of example only, will now be described with reference to the appended drawings. Accordingly, the preferred embodiments are described for convenience of reference and without limitation of the invention to embodiments described herein. The scope of the invention being defined by the claims appended hereto.

The invention will now be described in more detail with reference to non-limiting embodiments and figures.

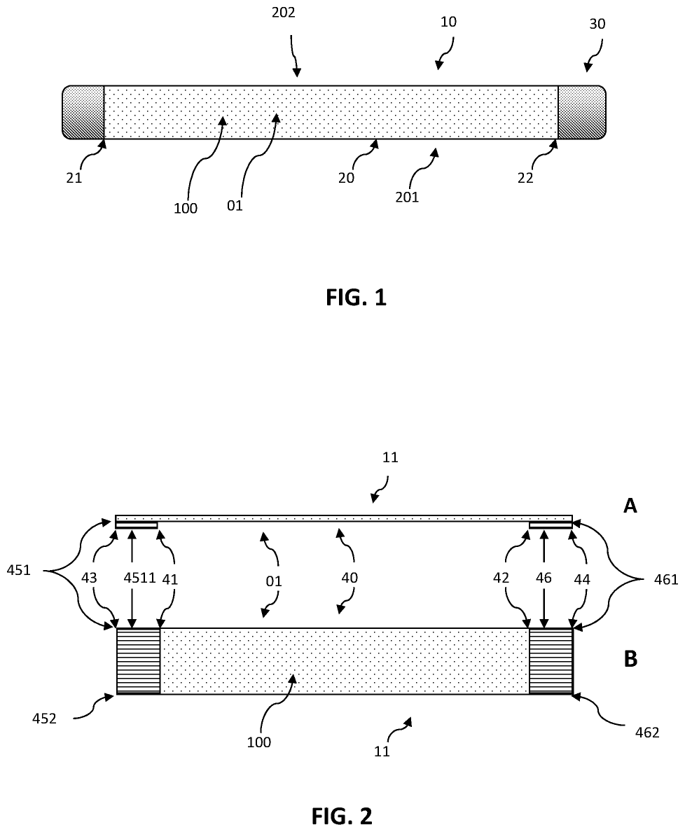

FIG. 1 shows an elastic medical implant encasement 10 placed onto a medical device, such as an orthopedic implant (e.g. an elongate bone plate) 30. As depicted, the encasement 10 can include a body 20, in this case elongated, having two ends 21, 22. One of ends 21, 22 is open and the other is open or closed, such that when at least one end is open, it allows the passage of at least part of the orthopedic implant to be placed therethrough. In one embodiment, both ends 21 and 22 are preferably open-ended, thereby allowing an implant to be inserted into encasement 10 from either side. In another embodiment, one of ends 21 or 22 is a closed end so that an implant may be inserted into encasement 10 from the open end only, thereby providing a pouch that grips the implant following insertion of the implant into the pouch/encasement 10. In the embodiments covered by FIG. 1, at least part of the body 20 and/or end 21 and/or end 22 is smaller than a complementary part the implant, such that the implant is held securely, or gripped, by the encasement following the insertion of the implant into the end(s) of the encasement. When the body is smaller than the implant, it will be understood that a gripping force may be supplied by the body due to elastic deformation of the body, as the body is prevented from relaxing by the anchorage provided by one or more of ends 20, 21 and/or the implant. More generally, the grip provided by the end(s) and, potentially, the rest of the body of the encasement results from the elasticity of the sheets used to make the encasement, which are stretched sufficiently to enable the implant to be inserted into the encasement and then the sheets recover towards their original size. The resulting grip provided by the encasement prevents the implant from sliding in relation to the encasement (or vice versa) when the implant is being implanted or affixed at the surgical site in a patient. It will be appreciated that the whole or part of the encasement 10 may include at least one biologically active agent as hereinbefore described, along with any necessary excipients or release agents.

FIG. 2A shows a side view of a further elastic encasement 11, again with bioactive agents loaded therein, according to the principles of the present invention. FIG. 2B shows the bottom view of the same embodiment shown in FIG. 2A.

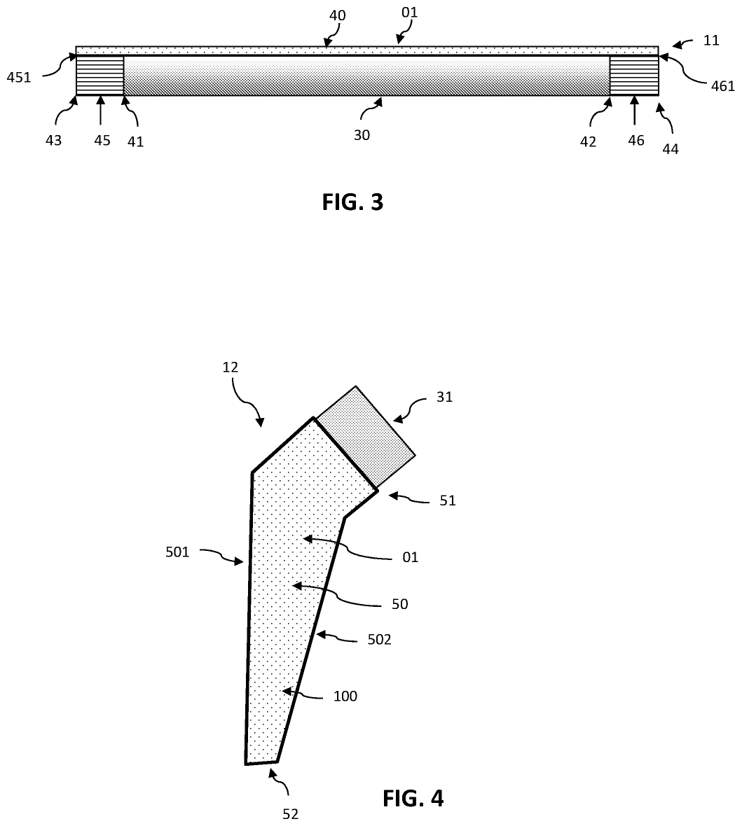

FIG. 3 shows the elastic encasement 11, of FIG. 2 with a medical device such as orthopedic implant 30 inserted therein, which in the non-limiting embodiment shown may be an elongate bone plate. Referring to FIGS. 2 and 3, a preferred embodiment of encasement 11 may include a body 40, in this case a film, for the purpose of only covering the implant on one side. The embodiment shown has two anchoring slots 45, 46 and four ends 41, 42, 43, 44. Ends 41, 42 may be open. Ends 43, 44 may be either open or closed. In one embodiment, anchoring slots 45, 46 may be formed from a single piece of the encasement by folding an elastic sheet onto itself and sealing along the edges 451, 452 for slot 45 and edges 461 and 462 for slot 46. In this embodiment, ends 43, 44 are the folded edge and they will naturally be closed, unless cut open. In an alternative embodiment, anchoring slots 45, 46 may be formed from at least 2 pieces of encasement grouped together to form the slot and sealed along the edges 451, 452 for slot 45 and edges 461 and 462 for slot 46. In this embodiment, ends 43, 44 can be left open without seal or they can be closed with seal. In yet a further alternative embodiment, anchoring slots 45, 46 may be formed from either one of the methods described above. In the embodiments, ends 41 and 42 are open ends, through which an implant may be inserted into the anchoring slot 45 and 46 respectively. In one embodiment, the length of the body 40 is shorter than the corresponding length of the implant, such that the body 40 is stretched when the implant is inserted and achieves a grip along the direction of the stretched length. In another embodiment, at least partial of the circumferences of the anchoring slot 45 and/or end 41 and/or end 43 and anchoring slot 46 and/or end 42 and/or end 44 is smaller than the corresponding circumference of the implant, such that a grip is achieved via the elasticity of the encasement along the radial direction of the stretched circumference. In another embodiment, the grip can be achieved via a combination of shorter body length and smaller anchoring slots. It will be appreciated that both the longitudinal and radial gripping effects may be combined in a single encasement embodiment. Embodiments of the kind provided by encasement 11 highlights the possibility that the encasement can be designed such that the drug is released from only one side of the implant to direct the drug into bone or the nearby soft tissue as desired.

FIG. 4 shows an elastic encasement 12, with bioactive agents loaded, according to principles of the present invention placed onto a medical device such as orthopedic implant 31, which in one non-limiting embodiment shown may be a hip implant. A preferred embodiment of an agent(s)-carrying encasement 12 may include a body 50, in this case odd-shaped, having two ends 51, 52. Ends 51, 52 may be either open or closed. In one embodiment, both ends 51 and 52 are open-end through which an implant may be inserted into encasement 50 preferably from end 51 due to its odd shape. In another embodiment, either one of the ends 51, 52 is a closed end so that an implant may be inserted into encasement 50 only from the open end and the rest of the edges are encased by 50. In these embodiments, at least part of the circumference of body 50 and/or end 51 and/or end 52 is smaller than the corresponding circumference of the implant, such that the implant is held with a gripping force by at least one portion of the encasement. As noted hereinbefore, the grip generated by the elasticity of the one or more sheets used to make the encasement can prevent the encasement from sliding in relation to the implant (or vice versa) when the implant is being implanted or affixed at the surgical site in the patient. In embodiments where the implant will have an odd shape, the encasement 12 may be shaped to follow the outline of the implant to enable a better conformity to be achieved. Encasement 50 highlights the ability of the elastic encasement to grip onto the implant tightly, without generating extra space between the implant and encasement. This is important for implants where there is limited space and/or where extra forces like shear force will be experienced during the implantation. Any other encasement, pouch, envelope, encasement etc. that cannot achieve the tight grip with tight conformity will render it not suitable for implant of this class.

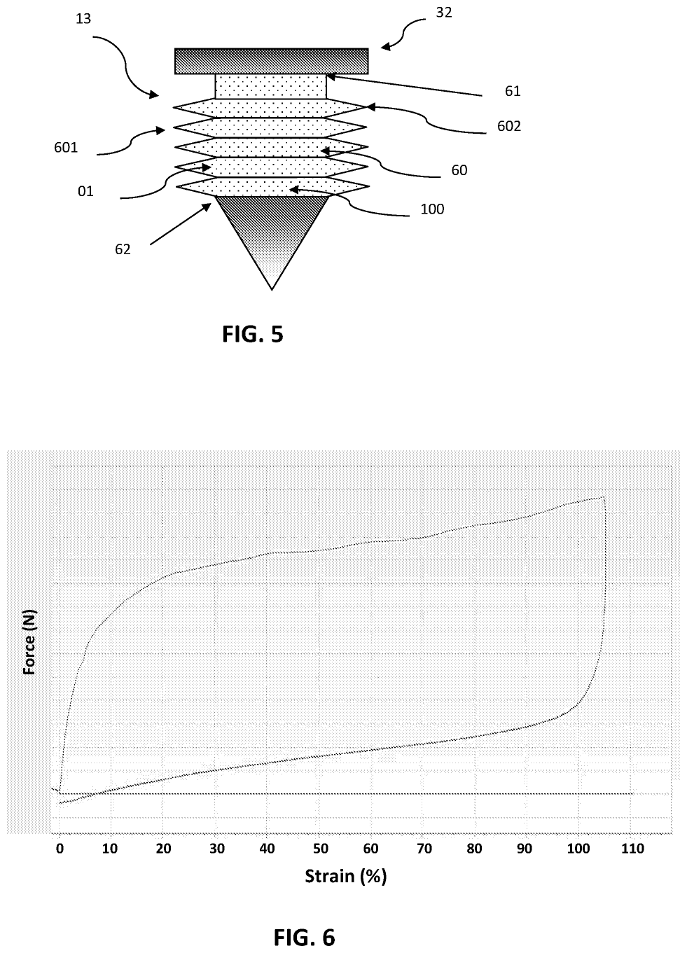

FIG. 5 shows an elastic encasement 13, with bioactive agents loaded, according to principles of the present invention placed onto a medical device such as orthopedic implant 32, which in the non-limiting embodiment shown may be an orthopedic screw or dental implant screw. Encasements 13 (FIG. 5) and 10 (FIG. 1) are essentially very similar and encasement 13 highlights the ability of the elastic encasement to retain the shape of the implant and thereby retain the essential functionality of the implant. Any other encasement, pouch, envelope, encasement etc. that cannot retain the implant shape will render it not suitable for implant of this class. A preferred embodiment of an agents-carrying encasement 13 may include a body 60, in this case cylindrical, having two ends 61, 62. One of ends 61, 62 is open and the other is either open or closed. In one embodiment, both ends 61 and 62 are open-end through which an implant may be inserted into encasement 13 from either side. In another embodiment, end 61 preferably is a closed end so that an implant may be inserted into encasement 13 from the open end only and the rest of the edges are gripped by 13. In the embodiments, at least part of the body 60 and/or end 61 and/or end 62 is smaller than the corresponding circumference of the implant, such that the implant is gripped tightly by the encasement, which prevents sliding of the implant/encasement relative to one another when the implant is being fitted, or thereafter.

In the embodiments, encasement 10, 11, 12, 13 may be formed from a single thin sheet or film 01 of a bioresorbable material, or more than one sheet. Film 01 in a preferred embodiment is made of a biodegradable resorbable polymer (as defined hereinbefore) which will dissolve away over time when implanted in vivo and be absorbed into a patient, leaving only the implant behind if the implant is not made of a resorbable material. Alternatively, the implant may also be made of a resorbable material in other embodiments in which case both the implant and encasement will eventually dissolve. Film 01 may be generally thin and substantially planar in a preferred embodiment, which may without limitation have a typical illustrative thickness T in a range from about 0.01 um to 1000 um, and more preferably in a range from about 0.04 mm to 0.2 mm. Any suitable sheet thickness T, however, may be used depending on the intended application, considerations for tear-resistance when inserting an implant into the encasement, drug dispensing duration, etc. Film 01 may be made by any suitable means known in the art. As noted hereinbefore, it is specifically contemplated that more than one sheet of elastic material can be used and these are contemplated here too.

Encasement 10, 11, 12, 13 may be made in one embodiment using a thermally processed, compression molded sheet of degradable polymer. In one embodiment, the drug or other biological agent may be dissolved or dispersed into the polymer while still in solution form. In one embodiment, the polymer solution is then processed into a film using conventional methods known in the art, perforated, and then fashioned into a encasement as described herein. Preferably, film 01 may be perforated by any suitable technique, such as using a press in one embodiment, while the film is still in a generally flat state.

In one embodiment, encasement 10, 12, 13 may be a seamless tubular structure. In another embodiment, encasement 10, 12, 13 may be formed from perforated film 01 by folding the film over itself to create a folded edge and sealing the opposite overlapping edges. In this case either edge 201, 202; 501, 502; 601, 602 is the folded edge while the corresponding opposite edge will be sealed to create a fused seam. In another embodiment, the encasement may be formed from at least 2 pieces of film 01 grouped together and sealed along the edges 201, 202; 501, 502; 601, 602. It should be noted that any suitable technique may be used to form a seal and close free edge, such as chemical fusion or welding, use of biologically compatible adhesives, etc. Accordingly, the invention is not limited to the use of heat fusion techniques. In addition, the seal need not be a full continuous seal.

In one embodiment of an elastic encasement, the resorbable polymer used for film 01 preferably contains poly(lactide-co-caprolactone) (PLCL) (e.g. having a PLA to PCL ratio of from 90:10 to 60:40) or its derivatives and copolymers thereof, and/or the bioresorbable elastomeric polymeric material of one of the at least one polymer layers is poly(DL-lactide-co-caprolactone) (DL-PLCL) (e.g. having a DL-PLA to PCL ratio of from 90:10 to 50:50) or its derivatives and copolymers thereof, and/or the bioresorbable elastomeric polymeric material of one of the at least one polymer layers is poly(glycolide-co-caprolactone) (PGCL) (e.g. having a PGA to PCL ratio of from 90:10 to 10:90) or its derivatives and copolymers thereof, or, more particularly, the bioresorbable elastomeric polymeric material of one of the at least one polymer layers may be a blend of PCL and PLA (e.g. a ratio blend of PCL and PLA having a wt:wt ratio of 1:9 to 9:1).

Encasements containing at least a portion of the preferred resorbable, flexible polymers (e.g., PLCL) advantageously have properties of good flexibility, elasticity and strength. In one embodiment, an elastic encasement is readily stretchable to conform to the size and shape of the implant, has sufficient strength to resist tearing during stretching of the encasement, and has sufficient grip strength to resist movement of the encasement in relation to the implant. In one preferred embodiment, sheet 01 preferably is capable of stretching up to at least 100% elongation of its initial unstretched length or width and return to its original size or to a size no greater than the expanded size minus (90% of the difference between expanded size and original size). Advantageously, a single elastic encasement may fit a wide range of implant sizes and/or shapes, and preferably provide a relatively snug fit and grip over the medical implant in a preferred embodiment, with or without slight modification by the surgeon as described herein. In one embodiment, the present invention includes a kit including a limited number of encasement of different sizes and/or shapes that may be able to fit over a majority of an implant product line.

In a preferred embodiment, encasement 10, 11, 12, 13 preferably further contains a plurality of apertures or perforations 100 of any suitable shape (such as substantially round perforations or apertures) in one possible embodiment to allow the passage or transport of fluids through the encasement. Perforations 100 need not be perfectly round, and may be ovoid or elliptical in shape in some embodiments (not shown). The apertures 100 are not limited to round perforations. Preferably, perforations 100 extend completely through sheet 01 from an inside surface to an outside surface. Perforations 100 can advantageously provide distribution of the drug or biological agent to adjacent tissue and bone in a preferred manner. In addition to benefiting drug distribution, perforations 100 can also enhances the stretchability of the encasement and improves ease of use and conformity. A preferred illustrative non-limiting range for porosity based on percentage of open area provided by perforations 100 to total surface area of film 01 is from about 10% to about 90%, and more preferably from about 20% to about 80%. Perforations 100 preferably have a diameter of at least about 0.1 mm for satisfactory drug distribution and flushing. In a preferred embodiment, perforations 100 have a diameter of at least about 1 mm. Diameters of approximately 0.1 mm or greater are generally considered in the art to represent macroporosity.

Encasement 10, 11, 12, 13 preferably is supplied separately in its own sterile pouch. The surgeon may use the encasement by removing the encasement from the pouch, and then sliding and stretching the encasement over an implant 30, 31 or 32. Implant may be slid into encasement to achieve a snug fit and avoid excessive unsupported loose encasement material on the end. Encasement 10, 12, 13 may be trimmed using a surgical scissors to remove excess encasement length to fit to length of the implant. It should be noted that although a somewhat snug fit between encasement and implant may be desired, a tight fit is not required in all instances. Similar techniques described above may be used by the surgeon to modify encasement 10, 11, 12, 13 for custom fitting the encasement to the particular size and shape of the implant needed to be encased. The implant encased within the encasement may then be implanted into the patient and fixed in place using standard methods. Advantageously, the surgeon will be able to deploy a drug from a variety of implants via the encasement, but medical device companies will avoid the onerous logistics of developing and maintaining large uncoated and coated implant inventories, with one or more drugs depending on the condition of the patient or indication to be treated.

It will be appreciated that numerous different shapes and types of medical implants may be used with the invention without limitation. Accordingly, Encasement 10, 11, 12, 13 may be used with devices other than bone plates hip implant and screws as shown, such as without limitation non-orthopedic implants (e.g., stents, pacemakers, dental implants, bone grafts etc.) and other orthopedic implants (e.g., tibia nails, femoral nails, spinal implants, etc.). Accordingly, in some embodiments, the surgeon may combine two or more encasements of the same or different sizes and shapes for an implant. For example, two or more encasement 10 may be combined without limitation for use with bone plates or other types of medical implants having an L-shape, T-shape, X-shape, H-shape or other types and shapes of implants. It should be recognized that the implant need not be completely encased by encasement in all cases to effectively deliver a drug or other biological agent to surrounding tissue.

While the description and drawings represent preferred embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, sizes, and with other elements, materials, and components, without departing from the spirit or essential characteristics thereof. One skilled in the art will appreciate that the invention may be used with many modifications of structure, arrangement, proportions, sizes, materials, and components used in the practice of the invention, which are particularly adapted to specific needs and operating requirements, without departing from the principles of the present invention. The presently disclosed embodiments are therefore to be considered in all respects as illustrative and not restrictive, the scope of the invention being defined by the appended claims, and not limited to the foregoing description or embodiments.

### EXAMPLES

### Example 1

Elasticity of Film

Samples of film where the film is made from poly(lactide-co-caprolactone) with a ratio range of from 90:10 to 60:40 (lactide:caprolactone) were subjected to tensile testing. FIG. 6 shows the load vs extension graph of a representative example of the tested films. The initial film length was 30 mm and the test shows a pulling of up to at least 100% elongation (i.e. stretched to 200% of its original length). FIG. 6 illustrates the corresponding force generated on the force gauge by the material at each elongation length and its recovery path due to the material's elasticity. FIG. 6 also illustrates the ability of the material to stretch beyond 100% elongation without experiencing failure and recover to near its original length at the end of the experiment. The experiment was conducted using a tensile tester CMT-6001.

Drug Elution from Films

The following examples are intended to demonstrate various layered films that may be used in the current invention and are not intended to be limiting in nature. These films may be used to create encasements for any medical implant that is in need thereof.

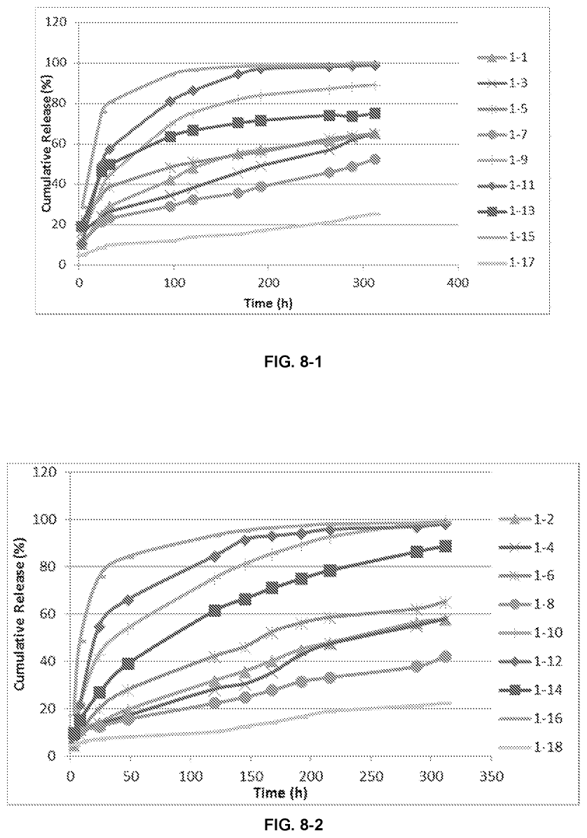

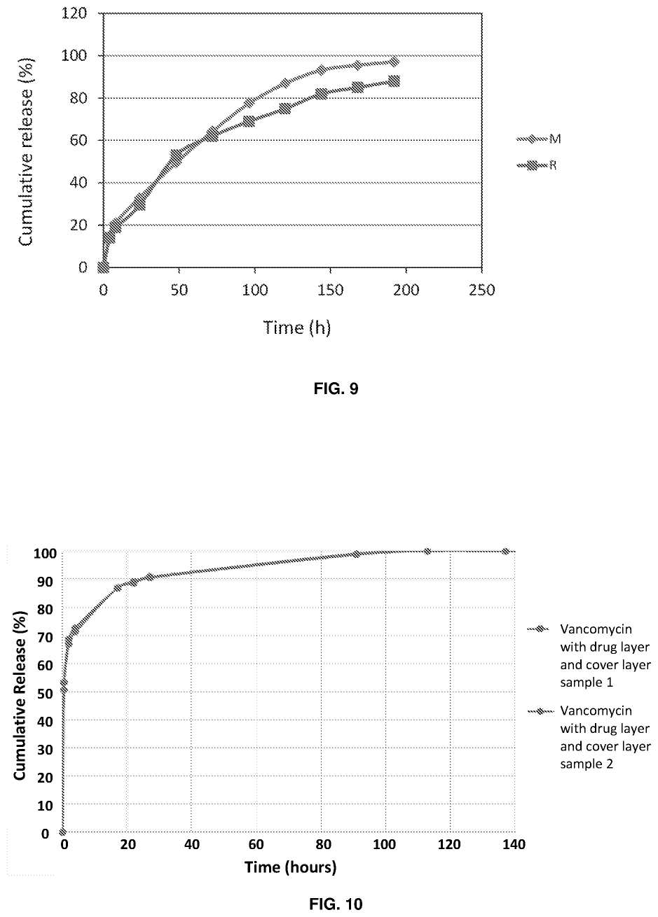

To illustrate the kinetics of drug release, a sample of a film was cut into a 2 cm×2 cm size, which was immersed in a vial containing 4 mL of PBS buffer (as the elution medium) for continuous drug elution testing. The vial was placed in a 37° C. incubator shaker. At periodic intervals, the elution medium was withdrawn for reverse phase HPLC analysis to determine the eluted amount of rifampicin and minocycline (or vancomycin alone) and replaced with fresh PBS solution (4 mL). The cumulative drug release was calculated and plotted (see FIGS. 8-9).

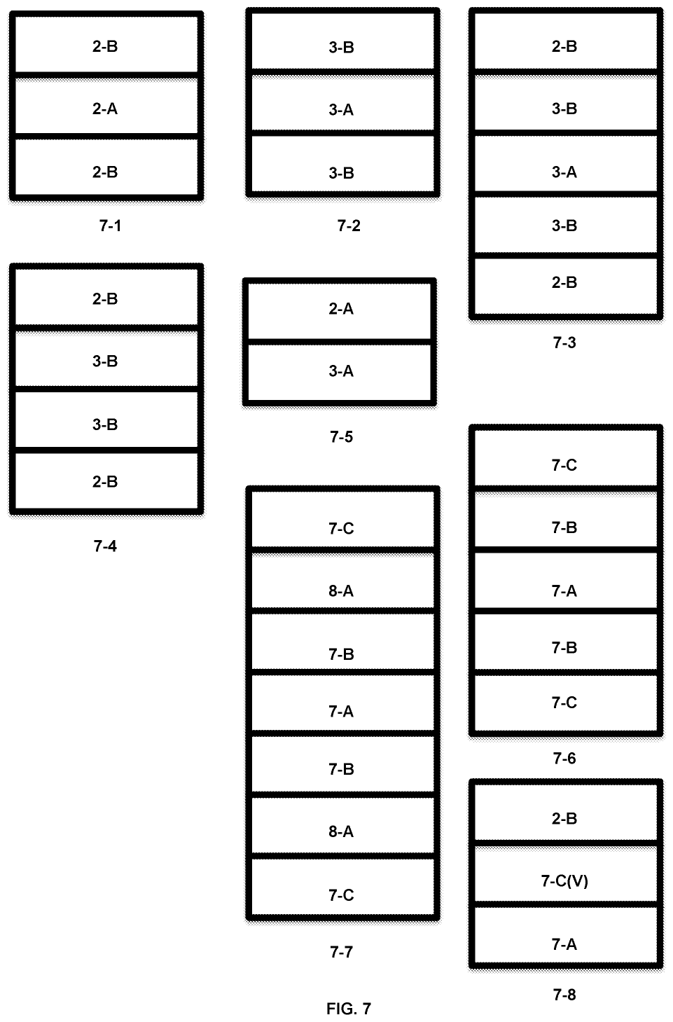

Table 1 and FIG. 7 list a series of designs that were used in the examples. The table lists a number of polymers that can be used to generate compositions according to the current invention (whether alone or in combination), as well as antibiotics. It will be understood that alternative polymers and antibiotics may be used.

TABLE 1
Film matrix with rifampin (R) and minocycline (M)

| Film code | Design | Polymer | Antibiotics |
|---|---|---|---|
| 1-1 | 4-1 | PLCL, PLA, PLGA | M |
| 1-2 | 4-1 | PLCL, PLA, PLGA | R |
| 1-3 | 4-2 | PLCL, PLA, PLGA | M |
| 1-4 | 4-2 | PLCL, PLA, PLGA | R |
| 1-5 | 4-3 | PLCL, PLA, PLGA | M |
| 1-6 | 4-3 | PLCL, PLA, PLGA | R |
| 1-7 | 4-4 | PLCL, PLA, PLGA | M |
| 1-8 | 4-4 | PLCL, PLA, PLGA | R |
| 1-9 | 4-5 | PLCL, PLA, PLGA | M |
| 1-10 | 4-5 | PLCL, PLA, PLGA | R |
| 1-11 | 4-6 | PLCL, PLA, PLGA | M |
| 1-12 | 4-6 | PLCL, PLA, PLGA | R |
| 1-13 | 4-7 | PLCL, PLA, PLGA | M |
| 1-14 | 4-7 | PLCL, PLA, PLGA | R |
| 1-15 | Single | PLCL | M |
| | Layer, | | |
| | with | | |
| | releasing | | |
| | agent | | |
| 1-16 | Single | PLCL | R |
| | Layer, | | |
| | with | | |
| | releasing | | |
| | agent | | |
| 1-17 | Single | PLCL | M |
| | Layer, | | |
| | without | | |
| | releasing | | |
| | agent | | |
| 1-18 | Single | PLCL | R |
| | Layer, | | |
| | without | | |
| | releasing | | |
| | agent | | |

### Example 2 (Design 7-1, Film Codes 1-1 and 1-2)

For the avoidance of doubt, “Design 7-1” refers to the design depicted by FIG. 7-1. All other references to “Designs” should be interpreted accordingly.

2-A Film Casting for Drug—Resorbable Film

1.8 g PLCL resin, 700 mg of sorbitol and 160 mg of minocycline (film code 1-1; rifampicin for film code 1-2) were dissolved in 10 mL acetone/ethanol solvent mixture of the ratio of 5:5 v/v. The mixture was mixed evenly for more than 4 hours. After the mixing, the solution was homogeneous and 5 mL of the solution was then poured onto a glass plate and drawn by a film applicator to form a film upon drying. The film was removed from the glass plate after the film was completely dry, following evaporation of the solvent.

2-B Film Casting for Control Layer Film

Similarly, 1.8 g PLCL resin and 50 mg sorbitol were dissolved in 10 mL of acetone. A homogeneous solution was poured onto a glass plate and drawn by a film applicator to form a film following evaporation of the solvent. The film was then removed from the glass plate. 2-C Films compression

A composition according to design 7-1 was prepared using two films according to 2-B sandwiching a film according to 2-A. The resulting stack of films were aligned and compressed by a heat compressor at 60° C., 6 MPa for 50 seconds.

### Example 3 (Design 7-2, Film Codes 1-3 and 1-4)

3-A Film Casting for Drug—Biodegradable Film

1.8 g PLCL/PLC resin (2:8 weight ratio) and 160 mg of minocycline (film code 1-3; rifampicin for film code 1-4) were dissolved in 10 ml acetone/ethanol solvent mixture having a ratio of 5:5 v/v. The film casting procedure was the same as described in Example 2-A.

3-B Spray Coating of Drug—PLGA Mixture

Similarly, 180 mg PLGA resin and 20 mg of minocycline (film code 1-3; rifampicin for film code 1-4) were dissolved in 10 ml acetone/ethanol solvent mixture having the ratio of 5:5 v/v. The mixture was spray coated onto the film prepared in 2-A, using 2 ml of the prepared solution, by repeatedly passing the spray nozzle over both sides of film 2-A with the same number of passes.

### Example 4 (Design 7-3, Film Code 1-5 and 1-6)

The middle three layers were prepared by following procedure in Example 3. The two outer layers were prepared by following Example 2-B. The stack of 5 layers of films were aligned properly and compressed by a heat compressor at 60° C., 6 MPa for 50 seconds.

### Example 5 (Design 7-4, Film Code 1-7 and 1-8)

The outer two layers were prepared by following Example 2-B. The two middle drug-polymer layers were prepared by following Example 3-B. The resulting films were aligned properly and compressed by a heat compressor at 60° C., 6 MPa for 50 seconds.

### Example 6 (Design 7-5, Film Code 1-9 and 1-10)

The two layers were prepared by following Example 2-A and 3-A. Film compression procedure is the same as 2-C.

### Example 7 (Design 7-6, Film Code 1-11 and 1-12)

7-A Film Compression for Elastic Biodegradable Polymer Film

PLCL resin was heat compressed at 150° C., 60 Mpa for 1 minute.

7-B Spray Coating of Drug—PLGA Mixture

180 mg PLGA resin and 20 mg of minocycline (film code 1-11; rifampicin for film code 1-12) were dissolved in 10 ml acetone/ethanol solvent mixture having the ratio of 5:5 v/v. The mixture was spray coated onto the film prepared in 7-A, using 2 ml of the prepared solution, by repeatedly passing the spray nozzle over both sides of film 7-A with the same number of passes.

7-C Film Casting for Blend of Small Molecules Drug Film

1.8 g PLCL resin, 250 mg of polysorbate and 160 mg of minocycline (film code 1-1; rifampicin for film code 1-2) were dissolved in 10 mL acetone/ethanol solvent mixture of the ratio of 5:5 v/v. The mixture was mixed evenly for more than 4 hours. After the mixing, the solution was homogeneous and 5 m L of the solution was then poured onto a glass plate and drawn by a film applicator to form a film upon drying. The film was removed from the glass plate after the film was completely dry, following evaporation of the solvent.

7-D Films Compression

A composition according to design 7-6 was prepared using two films according to 7-C sandwiching a film 7-A coated according to 7-B. The resulting stack of films were aligned and compressed by a heat compressor at 60° C., 6 MPa for 50 seconds.

### Example 8 (Design 7-7, Film Code 1-13 and 1-14)

8-A Film Casting for Blend of Small Molecule Control Film

1.8 g PLCL resin and 50 mg of polysorbate were dissolved in 10 ml acetone/ethanol solvent mixture of the ratio of 5:5 v/v. The mixture was mixed evenly for more than 4 hours. After the mixing, the solution was homogeneous and 5 ml of the solution was then poured onto a glass plate and drawn by a film applicator to form a film upon drying. The film was removed from the glass plate after the film was completely dry, following evaporation of the solvent.

8-B Films Compression

A composition according to design 7-7 was prepared using two films according to 8-A sandwiching a film 7-A coated according to 7-B. The stack is further sandwiched between two films according to 7-C. The resulting stack of films were aligned and compressed by a heat compressor at 60° C., 6 MPa for 50 seconds.

### Example 9 (Single Layer, with Releasing Agent, Film Code 1-15 and 1-16)

Film preparation procedure is the same as Example 2-A to prepare a single layer.

### Example 10 (Single Layer without Releasing Agent, Film Code 1-17 and 1-18)

10-A Film Casting for Drug—Resorbable Film

0.5 g PLCL resin and 160 mg of minocycline (film code 1-1; rifampicin for film code 1-2) were dissolved in 10 mL acetone/ethanol solvent mixture of the ratio of 5:5 v/v. The mixture was mixed evenly for more than 4 hours. After the mixing, the solution was homogeneous and 5 m L of the solution was then poured onto a glass plate and drawn by a film applicator to form a film upon drying. The film was removed from the glass plate after the film was completely dry, following evaporation of the solvent.

### Example 11 (Mixed Drug)

The film was prepared by following the protocol in Example 4. The middle layer was prepared by using a drug mixture of 120 mg minocycline and 160 mg rifampin. The two intermittent layers were prepared by spray coating of minocycline by following Example 3-B.

The outer two layers were prepared by following Example 2-B. The stack of 5 layers of films were aligned properly and compressed by a heat compressor at 60° C., 6 MPa for 50 seconds. The cumulative releasing profiles of two antibiotics are shown in FIG. 9.

### Example 12

FIG. 8 shows the cumulative release of two antibiotics from different layered film designs and single films prepared in Examples 2 to 10 (film codes 1-1 to 1-18). The drug density of both antibiotics is between 0.05 mg to 0.1 mg/cm2. As shown in FIG. 8, for single drug film, the absence of releasing agent results in a film with very slow release, while the presence of releasing agent gives a high initial burst with a fast releasing profile. Because minocycline is more hydrophilic than rifampin, minocycline releases much faster. For the layered film designs, the release profile and initial burst rate of rifampin and minocycline are tuned and well-controlled through the different designs.

This result shows that by knowing the releasing behaviour of each drug in the different designs, the releasing profile of a drug mixture can be tuned to provide a desired releasing profile. This can be clearly seen from FIG. 9, which shows a significant improvement from literature data where rifampin always has a lower initial burst and slower releasing profile than the other hydrophilic counterpart (in this case minocycline).

### Example 13

FIG. 10 shows the cumulative release of the antibiotic vancomycin from a further layered film design (7-8). In this example, a first film layer is prepared in accordance with the method of 7-A above, on top of which is placed a film layer prepared in accordance with 7-C above with the exception that minocycline is replaced by vancomycin. Finally, a layer prepared in accordance with 2-B is placed on top of the vancomycin-containing layer and the three layers are then compressed together using the method of 2-C as disclosed above to form the product.

As shown in FIG. 10, the product shows a very consistent release between the replicates. The release experiment was conducted in accordance with the procedure set out in the section entitled “Drug Elution From Films” hereinbefore.

### Example 14

The zone-of-inhibition (ZOI) for the film was determined according to the Kirby-Bauer method. The study chose to test Escherisia coli (E. coli) and S. aureus, S.epidermidisas demonstration. E. coli has the highest minimum inhibitory concentration (MIC) among the other bacteria that are commonly found in humans. The MIC of E. coli is 20 times higher than S. aureus, S.epidermidis, MRSA, S. capitis etc.

E. coli were inoculated into Lysogeny broth (LB broth) from a stock solution and incubated at 37° C. and then evenly spread over the entirety of an agar plate by a disposable spreader. A 15 mm diameter film was firmly pressed into the center of an agar plate and incubated at 37° C. Pieces were transferred to other fresh agar plates using sterile forceps every 24 hr. The diameter of the ZOI was measured and recorded every day.

TABLE 2
ZOI of layer-by-layer composite film with minocycline and rifampicin.

| | E. Coli | S. epidermidis | S. aureus |
|---|---|---|---|
| Day 1/mm | 30.0 | 47.3 | 37.3 |
| Day 2/mm | 25.8 | 41.0 | 37.0 |
| Day 3/mm | 23.8 | 39.0 | 36.3 |
| Day 4/mm | 21.5 | 42.0 | 33.3 |
| Day 5/mm | 18.3 | 34.0 | 31.3 |
| Day 6/mm | 16.4 | 34.0 | 27.5 |
| Day 7/mm | 15.8 | 33.8 | 26.8 |
| Day 8/mm | No Zone | 32.4 | 26.3 |
| Day 9/mm | | 31.0 | 26.8 |
| Day 10/mm | | 29.9 | 25.8 |
| Day 11/mm | | 28.7 | 25.0 |
| Day 12/mm | | 26.5 | 24.5 |
| Day 13/mm | | 25.0 | 21.0 |
| Day 14/mm | | 23.9 | 20.3 |

I/We claim:

1. An elastic medical implant encasement, comprising:
at least one sheet of elastic material configured to form an encasement for at least part of a medical implant; and
at least one biologically active substance in at least one region of the at least one sheet of elastic material, wherein
the at least one sheet of elastic material comprises at least one polymer that is biologically-compatible and resorbable and has an elastic recovery of from 80% to 100% following stretching, or can stretch from its original size to an expanded size and return to its original size or to a size no greater than the expanded size minus 80% of the difference between expanded size and original size, optionally wherein the encasement or film can stretch from its original size to an expanded size and return to its original size or to a size no greater than the expanded size minus 90% of the difference between expanded size and original size.

2. The encasement of claim 1, wherein the encasement is in the form of a tube, an envelope, a body comprising one or more anchoring portions, a film comprising two or more anchoring points, or a combination of any of these forms.

3. The encasement of claim 1 or claim 2, wherein the encasement comprises:
(a) at least one sheet of elastic material with two or more anchoring points formed via folding onto itself or with at least one additional sheet of elastic material, where at least one of the elastic material sheets carries at least one biologically active substance in at least one region;
(b) at least one sheet of elastic biologically-compatible, resorbable, material folded onto itself to form a single large anchoring surface, where the at least one elastic material sheet carries at least one biologically active substance in at least one region;
(c) at least two sheets of elastic material sealed at overlapping areas to form one or more anchoring points or surfaces, where at least one of the elastic material sheets carries at least one biologically active substance in at least one region; or
(d) the encasement comprises a seamless tubular structure formed from at least one sheet of elastic material, where the at least one elastic material sheet carries at least one biologically active substance in at least one region.

4. The encasement of any one of the preceding claims, wherein the biologically active substance is encapsulated within the at least one sheet of elastic material and/or is coated on the surface of the at least one sheet of elastic material.

5. The encasement of any one of the preceding claims, wherein the at least one sheet of elastic material is from two to ten sheets of elastic material.

6. The encasement of claim 5, wherein the biologically active substance is encapsulated within one or more of the two to ten sheets of elastic material and/or is coated on the surface of one or more of the two to ten sheets of elastic material, optionally wherein the coated surface is not an outer surface of the two to ten sheets of elastic material.

7. The encasement of any one of the preceding claims, wherein the one or more elastic sheets are configured to release the at least one biologically active substance at at least one releasing rate.

8. The encasement of any one of the preceding claims, wherein the at least one sheet of elastic material has a total thickness of from 0.01 μm to 1000 μm.

9. The encasement of any one of the preceding claims, wherein the at least one polymer is selected from one or more of the group consisting of poly(lactide-co-caprolactone), poly(DL-lactide-co-caprolactone) (DL-PLCL), poly(L-lactide-co-caprolactone) (PLLCL)polycaprolactone (PCL), polyglycolide (PGA), poly(L-lactic acid) (PLLA), poly(glycolide-co-caprolactone) (PGCL) copolymer, poly(D,L-lactic acid), poly(L-lactide-co-D,L-lactide) (PLDLLA), poly(L-lactide-co-glycolide) (PLGA), poly(D,L-lactide-co-glycolide), poly (D-lactide) (PDLA), poly(trimethylene carbonate) (PTMC), poly(lactide-co-trimethylene carbonate) (PLTMC), poly(gycolide-trimethylene carbonate), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone-co-butylacrylate, a copolymer of polyhydroxybutyrate, a copolymer of maleic anhydride, a copolymer of poly(trimethylene carbonate), polyethylene glycol, hydroxypropylmethylcellulose and cellulose derivatives, polysaccharides, such as hyaluronic acid, chitosan, starch, proteins such as gelatin, collagen or PEG derivatives.

10. The encasement of any one of the preceding claims, wherein the number average molecular weight of the polymer is greater than 10,000 Daltons.

11. The encasement of any one of the preceding claims, wherein
a) the at least one polymer is poly(lactide-co-caprolactone) (PLCL) (e.g. having a PLA to PCL ratio of from 90:10 to 60:40) or its derivatives and copolymers thereof; and/or
b) the at least one polymer is poly(DL-lactide-co-caprolactone) (DL-PLCL) (e.g. having a DL-PLA to PCL ratio of from 90:10 to 50:50) or its derivatives and copolymers thereof; and/or
c) the at least one polymer is poly(glycolide-co-caprolactone) (PGCL) (e.g. having a PGA to PCL ratio of from 90:10 to 10:90) or its derivatives and copolymers thereof; and/or
d) the at least one polymer is a blend of PLCL or DL-PLCL or PGCL with a releasing agent selected from one or more of the group selected from polysorbate 20, polysorbate 40, polysorbate 60, polysorbate 80, or polyethyleneglycol having a molecular weight of 200 to 2000 Daltons in a w wt ratio of PLCL or DL-PLCL or PGCL to releasing agent of from 25:1 to 1:9.

12. The encasement of any one of the preceding claims, wherein the biologically active substance is selected from one or more of the group consisting of an adrenocorticostatic, a β-adrenolytic, an androgen or antiandrogen, an antianemic, an antiparasitic, an anabolic, an anaesthetic or analgesic, an analeptic, an antiallergic, an antiarrhythmic, an anti-arteriosclerotic, an antibiotic, an antidiabetic, an antifibrinolytic, an anticonvulsive, an angiogenesis inhibitor, an anticholinergic, an enzyme, a coenzyme or a corresponding inhibitor, an antihistaminic, an antihypertensive, an antihypotensive, an anticoagulant, an antimycotic, an antiseptic, an antiinfective, an antihemorrhagic, a betareceptor and calcium channel antagonist, an antimyasthenic, an antiphlogistic, an antipyretic, an antirheumatic, an antiseptic, a cardiotonic, a chemotherapeutic, a coronary dilatator, a cytostatic, a glucocorticoid, a haemostatic, an immunoglobuline or its fragment, a chemokine, a cytokine, a prodrug of a cytokines, a mitogen, a physiological or pharmacological inhibitor of mitogens, a cell differentiation factor, a cytotoxic agent and prodrugs thereof, a hormone, an immunosuppressant, an immunostimulant, a mineralcorticoid, a morphine antagonist, a muscle relaxant, a narcotic, a vector, a peptide, a (para)sympathicomimetic or (para)sympatholytic, a protein, a cell, a selective estrogen receptor modulator (SERM), a sedating agent, a spasmolytic, a substance that inhibits the resorption of bone, a vasoconstrictor or vasodilatator, a virustatic, and a wound healing substance.

13. The encasement of claim 12, wherein the biologically active substance is selected from one or more of the group consisting of an androgen or antiandrogen, an anaesthetic or analgesic, an antibiotic, an antiarrhythmic, an anti-arteriosclerotic, an antifibrinolytic, an angiogenesis inhibitor, an anticholinergic, an enzyme, a coenzyme or a corresponding inhibitor, an antihypertensive, an antihypotensive, an anticoagulant, an antimycotic, a betareceptor and calcium channel antagonist, an antiphlogistic, a coronary dilatator, a cytostatic, a glucocorticoid, a haemostatic, an immunoglobuline or its fragment, a chemokine, a cytokine, a prodrug of a cytokines, a mitogen, a physiological or pharmacological inhibitor of mitogens, a cell differentiation factor, a cytotoxic agent and prodrugs thereof, a hormone, an immunosuppressant, a mineralcorticoid, a morphine antagonist, a vector, a peptide, a protein, a cell, a selective estrogen receptor modulator (SERM), a sedating agent, a spasmolytic, a substance that inhibits the resorption of bone, a vasoconstrictor or vasodilatator, a virustatic, and a wound healing substance.

14. The encasement of claim 13, wherein the biologically active substance is selected from one or more of the group consisting of:
(a) an antimicrobial agent or an antifungal agent (e.g. the antimicrobial agent may be selected from one or more of the group consisting of tetracycline and its derivatives (such as minocycline, tigecycline and doxycycline), rifampin, triclosan, chlorhexidine, penicillins, aminoglycides, quinolones, vancomycin, gentamycine, tobramycin, a cephalosporin (e.g. cephalosporin), carbapenems, imipenem, ertapenem, an antimicrobial peptide, cecropin-mellitin, magainin, dermaseptin, cathelicidin, a-defensins, a-protegrins and pharmaceutically acceptable salts thereof (e.g. a combination of rifampin and another antimicrobial agent, such as a combination of rifampin and a tetracycline derivative), the antimicrobial agent may be a combination of rifampin and one or more of the group selected from minocycline, doxycycline, and tigecycline (e.g. rifampin and doxycycline, rifampin and tigecycline or, more particularly, rifampin and minocycline, such as a combination of rifampin and/or minocycline, for example, a combination of rifampin and minocycline, the ratio of rifampin to minocycline is from 1:10 to 10:1 (wt/wt) (e.g. from 2:5 to 5:2 (wt/wt)), the antifungal agent may be selected from one or more of the group consisting of azoles (such as ketoconazole, clotrimazole, miconazole, econazole, itraconazole, fluconazole, bifoconazole, terconazole, butaconazole, tioconazole, oxiconazole, sulconazole, saperconazole, clotrimazole, voriconazole, clotrimazole), allylamines (such as terbinafine), morpholines (such as amorolfine and naftifine), griseofulvin, haloprogin, butenafine, tolnaftate, nystatin, cyclohexamide, ciclopirox, flucytosine, terbinafin, amphotericin B and pharmaceutically acceptable salts thereof;
(b) anti-thrombotic agents such as heparin, heparin derivatives, urokinase, and PPack (dextrophenylalanine proline arginine chloromethylketone);
(c) anti-inflammatory agents such as dexamethasone, prednisolone, corticosterone, budesonide, estrogen, sulfasalazine and mesalamine;
(d) anesthetic agents such as lidocaine, bupivacaine and ropivacaine;
(e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides;
(f) vascular cell growth promoters such as hyaluronic acid, growth factors (Ciliary neurotrophic factor, fibroblast growth factors, hepatocyte growth factor, bone morphogenetic proteins), transcriptional activators, and translational promotors;
(g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
(h) protein kinase and tyrosine kinase inhibitors (e.g., tyrphostins, genistein, quinoxalines);
(i) cytotoxic agents, cytostatic agents and cell proliferation affectors;
(j) vasodilating agents;
(k) agents that interfere with endogenous vasoactive mechanisms;
(l) inhibitors of leukocyte recruitment, such as monoclonal antibodies;
(m) bone morphogenetic proteins, such as cytokines and metabologens;
(n) hormones;
(o) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin;
(p) alpha receptor antagonist (such as doxazosin, Tamsulosin) and beta receptor agonists (such as dobutamine, salmeterol), beta receptor antagonist (such as atenolol, metaprolol, butoxamine), angiotensin-II receptor antagonists (such as losartan, valsartan, irbesartan, candesartan and telmisartan), and antispasmodic drugs (such as oxybutynin chloride, flavoxate, tolterodine, hyoscyamine sulfate, diclomine);
(q) bARKct inhibitors;
(r) phospholamban inhibitors;
(s) Serca 2 gene/protein; and
(t) immune response modifiers including aminoquizolines, for instance, imidazoquinolines such as resiquimod and imiquimod.

15. The encasement of any one of the preceding claims, wherein the at least one elastic sheet may further comprise holes, optionally wherein the diameter of each of the holes is from 0.1 mm to 5 mm (e.g. from 0.3 mm to 2 mm), optionally:
(i) the shape of the holes are uniform and/or the holes are circular; and/or
(ii) the size of the holes are not uniform; and/or
(iii) the holes on the band are evenly distributed throughout the band, focused in the middle (avoiding seals) or nearer to the seals.

16. The encasement of any one of the preceding claims, wherein the encasement is selected from the group consisting of a pacemaker encasement, or, more particularly, an orthopedic implant encasement, a dental implant encasement, a simulator/sensory implant encasement, a subcutaneous implant encasement, a monitoring implant (e.g. biosensor chip) encasement, a breast implant encasement, an intra-uterine device encasement, an ear tubes (tympanostomy tube) encasement, and a tubing (e.g. catheters) encasement, where the encasement covers at least part of said implant.

17. The encasement of claim 16, wherein at least a portion of the encasement is dimensionally smaller than the implant to which it is to be applied to and which portion provides a gripping force when the encasement is applied to said implant.

18. The encasement of any one of the preceding claims, wherein the at least one sheet of elastic material has an elastic recovery of from 80% to 100% (e.g. from 85% to 100%, from 90% to 100% or from 95% to 100%) following stretching up to 300% (e.g. stretching to 100%) elongation and comprises at least one polymer that is biologically-compatible and resorbable.

19. A method of forming elastic medical implant encasement, comprising
(a) providing at least one sheet of an elastic material that further comprises at least one biologically active substance in at least one region of the sheet; and
(b) forming the at least one sheet into the elastic medical implant encasement.

20. The method of claim 19, wherein the method comprises:
(A)
(i) providing one sheet of an elastic material that further comprises at least one biologically active substance in at least one region of the sheet;
(ii) folding at least a part of the sheet onto itself to form an edge; and
(iii) sealing at least part of the edge to form the elastic medical implant encasement; and/or
(B)
(i) providing at least two sheets of elastic material where at least one of the sheets further comprises at least one biologically active substance in at least one region of said sheet;
(ii) overlapping the at least two sheets in at least one area to form an overlapping area; and
(iii) sealing at least part of the overlapped area to form the elastic medical implant encasement; and/or
(C)
providing at least one seamless tubular structure of elastic material having at least one biologically active substance in at least one region.

21. The method of claim 19 or claim 20, wherein forming and/or sealing is accomplished using one or more of the methods selected from the group consisting of heat fusion, chemical fusion, and adhesives.

22. A medical implant at least partly covered with an elastic medical implant encasement according to any one of claims 1 to 18.

23. The medical implant of claim 22, wherein the medical implant is selected from the group consisting of a pacemaker, or, more particularly, an orthopedic implant, a dental implant, a simulator/sensory implant, a subcutaneous implant, a monitoring implant (e.g. biosensor chip), a breast implant, an intra-uterine device, an ear tube (tympanostomy tube), and a tubing (e.g. catheters).
